(19) United States
(12) Patent Application Publication
(10) Pub. No.: US 2020/0354445 A1
(43) Pub. Date: Nov. 12, 2020

(54) METHODS AND COMPOSITIONS FOR THE IMPROVEMENT OF LYSOSOMAL FUNCTION AND TREATMENT OF NEURODEGENERATIVE DISEASE

(71) Applicant: VANDERBILT UNIVERSITY, Nashville, TN (US)

(72) Inventor: Laura Lee DUGAN, Brentwood, TN (US)

(21) Appl. No.: 16/764,431

(22) PCT Filed: Nov. 15, 2018
(86) PCT No.: PCT/US2018/061281

§ 371 Date: May 15, 2020

Related U.S. Application Data
(60) Provisional application No. 62/586,527, filed on Nov. 15, 2017.

(51) Int. Cl. C07K 16/24

(52) U.S. Cl. CPC C07K 16/248; A61K 45/06

(57) ABSTRACT

Disclosed are methods of reducing age-dependent lysosome impairment and/or treating an age related neurodegenerative disease, fibrotic disease, or rheumatological disease in a subject comprising administering to the subject a therapeutic agent; wherein the therapeutic agent inhibits nicotinamide adenine dinucleotide phosphate (NADPH)-oxidase (NOX) or down-stream NOX effector.

This application claims the benefit of U.S. Provisional Application No. 62/586,527, filed on Nov. 15, 2017, which is incorporated herein by reference in its entirety.

This work was with government support under Grant No. RO1 AG033679 awarded by the National Institutes of Health. The government has certain rights in the invention.

### I. BACKGROUND

Age-related diseases are arguably the single greatest challenge for biomedicine in the 21ₛₜ Century. The number of Americans over age 65 will be 70 million by 2030-nearly 25% of the populations. An ever-increasing number of these older adults will be facing chronic progressive neurodegenerative conditions such as Parkinson's disease, Alzheimer's disease, and other dementing disorders. Age, itself, is by far the single greatest common risk factor for all of these late-onset neurodegenerative diseases. These demographics showing increasing prevalence of neurodegenerative diseases suggests an extremely large and expanding market for treatments. However, the mechanism(s) which confer this age-associated risk remain poorly understood and to date, there are no disease modifying therapies for any of these diseases.

There are symptomatic treatments for Parkinson's but these fail after sustained treatment due to continued progression of the disease. Treatments used for Alzheimer's are expensive, but essentially ineffective, serving primarily as a placebo for the family Any treatment that even slows any of these diseases in a functionally significant manner would be major step forward. What are needed are new effective treatments for neurodegenerative diseases.

### II. SUMMARY

Disclosed herein are methods of reducing age-dependent lysosome impairment, improving lysosome function, increasing initiation of autophagy, and/or treating an age related neurodegenerative disease, fibrotic disease, or rheumatological disease in a subject comprising administering to the subject a therapeutic agent; wherein the therapeutic agent inhibits nicotinamide adenine dinucleotide phosphate (NADPH)-oxidase (NOX) or downstream NOX effector.

In one aspect, disclosed herein are methods of reducing age-dependent lysosome impairment, improving lysosome function, increasing initiation of autophagy, and/or treating an age related neurodegenerative disease, fibrotic disease, or rheumatological disease of any preceding aspect, wherein the neurodegenerative disease comprises Alzheimer's disease, Parkinson's disease, Huntington's disease, spinocervellar ataxia type 1, age-related dementia, lewy body dementia, probably vascular dementia, frontotemporal dementia, and amyotrophic lateral sclerosis (ALS).

Also disclosed herein are methods of reducing age-dependent lysosome impairment, improving lysosome function, increasing initiation of autophagy, and/or treating an age related neurodegenerative disease, fibrotic disease, or rheumatological disease of any preceding aspect, wherein the NOX is a NOX isoform selected from NOX1, NOX2, NOX3, NOX4, or NOX5 and/or the downstream NOX effector comprises reactive oxygen species, hydrogen peroxide, or superoxide. Thus, in one aspect, the therapeutic agent can reduce age-dependent lysosome impairment, improve lysosome function, increase initiation of autophagy, and/or treat an age related neurodegenerative disease, fibrotic disease, or rheumatological disease by inhibiting, reducing, and/or eliminating a downstream NOX effector such as, for example, ROS, hydrogen peroxide, and/or superoxide.

In one aspect, disclosed herein are methods of reducing age-dependent lysosome impairment, improving lysosome function, increasing initiation of autophagy, and/or treating an age related neurodegenerative disease, fibrotic disease, or rheumatological disease of any preceding aspect wherein the therapeutic agent is a signal transducer and activator of transcription 3 (STAT3) inhibitor, and wherein the therapeutic agent reduces STAT3 mediated NOX activation, thereby causing NOX inhibition.

Also disclosed herein are method of reducing age-dependent lysosome impairment, improving lysosome function, increasing initiation of autophagy, and/or treating an age related neurodegenerative disease, fibrotic disease, or rheumatological disease of any preceding aspect, wherein the therapeutic agent is an anti-IL-6 antibody or anti-inflammatory agent, and wherein the therapeutic agent reduces IL-6 mediated NOX activation, thereby causing NOX inhibition.

Also disclosed herein are method of reducing age-dependent lysosome impairment, improving lysosome function, increasing initiation of autophagy, and/or treating an age related neurodegenerative disease, fibrotic disease, or rheumatological disease of any preceding aspect, wherein the agent that inhibits NOX or a NOX downstream effector is a small molecule, antibody, siRNA, antisense oligonucleotide, peptide, or protein including, but not limited to a small molecule comprising diphenylene iodonium. Apocynin, 2-(2-chlorophenyl)-4-[3-(dimethylamino)phenyl]-5-methyl-1H-pyrazolo[4,3-c]pyridine-3,6-dione (GKT-831), Rapamycin, dismutazyme, a malonic acid derivative of C60, an acetic acid derivative of C60, or any combination thereof.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, and 1D show Age-dependent accumulation of p62 aggregates in WT mouse hippocampus. Sections from young old (1A, 24 month) or young (1B, 4 month) mice were immunostained for p62 (red). FIG. 1C shows the % area of hippocampus occupied by the clusters, mean±SD, n=8, *p<0.05 by t-test. FIG. 1D shows 3-D reconstruction of an aggregate using Imaris software, showing hundreds of discrete 3-8 μm deposits within the aggregate. A 90° optical section through the middle of the aggregate is shown at bottom. Dapi (blue) was included to show cell nuclei.

FIGS. 2A, 2B, 2C, 2D, 2E, 2F, 2G, and 2H show Colocalization of p62-positive aggregates with other proteins. FIGS. 2A, 2B, 2C, 2D, and 2E show p62 (red) and parkin (green) immunostaining of old hippocampus. FIG. 2A shows merged channels, (2B) individual channels, (2C) Higher magnification of an aggregate (2D), 300 orthogonal view, (2E) Statistical correlation between signals (r2=0.81, p=0.013), using Sigmaplot 11 and Sigmastat 4.0. FIGS. 2F and 2G show merged images of p62 and Sumo3 (2F), or lamp2 (2G) (proteins also implicated in protein processing through lysosomes). FIG. 2H shows p62 and reelin (neuronal protein). There is substantial overlap for p62 and Sumo3 and Reelin, but only partial for lamp2.

FIGS. 3A, 3B, 3C, 3D, 3E, and 3F show partial overlapping between p62 positive protein aggregates and GABAergic neuronal fibers. Most p62 protein aggregates were not associated with calretinin (CR) (3A) and PV neurons (3D). Some p62 aggregates were labeled with CR (3B and 3C) or PV (3E and 3F). FIGS. 3B, 3C, 3E and 3F were confocal pictures of 1 mm thick optical sections. Arrowheads and arrows depict p62 puncta that were and were not labeled by CR or PV, respectively.

FIGS. 4A, 4B, 4C, 4D, 4E, 4F, 4G, and 4H show the association between astrocytes and p62 protein aggregates. p62 immunoreactivity was rarely observed in microglia (labeled by Iba; 4A and 4E). Some astrocytes, labeled by GFAP, contained p62 positive dots in the cell bodies. Occasionally, an astrocyte was surrounded by p62 positive aggregates (4B and 4F). The association of p62 containing aggregates with astrocytes was further confirmed in GFAP-GFP mice that enable visualization of fine projections of astrocytes with GFP fluorescence (4C and 4G). Similarly, reelin positive aggregates were also associated with or inside astrocytes (4D and 4H). FIGS. 4A, 4B, 4C, and 4D were projections of 5 to 20 sections of 1 mm thickness, while FIGS. 4E, 4F, 4G, and 4H were corresponding representatives of 1 μm thick sections to confirm whether there was colocalization.

FIG. 5 shows that Nox inhibition, or reduction of superoxide by a dismutase mimetic, C3, lessons the accumulation of p62 aggregates. Female C57BL6 mice received apocynin (50 mg/kg) or C3 (1 mg/kg/day) in their water from 12-17 mos of age, then perfused. Fixed brain were sectioned, and immunostained for p62. Confocal images were analyzed for 6 hippocampi/three sections per mouse, 4 mice per group. To quantify the p62-containing aggregates, aggregates were individually circled in the depicted region by a blinded investigator, and aggregate areas measured using ImageJ. Areas covered by aggregates, and number of clusters, are shown. Mean±SEM, *p<0.05, ***p<0.001, Mann-Whitney Rank Sum test.

FIG. 6 shows that lysosomes are significantly larger in hippocampal neurons from old mice compared to young mice. Brain sections from 5 month and 20 month old female mice were immunostained with antibodies against lamp1 and parvalbumin (PV), chosen to allow lysosomes in individual neurons to be analyzed. In old versus young animals, lamp1-labeled lysosomes are larger in both PV neurons as well as surrounding pyramidal neurons. Lysosomes in PV neurons were quantified in 1 μm thick optical sections with ImageJ using Analyze Particles function; n=8 young, n=7 old. *p<0.001, Mann-Whitney Rank Sum test.

FIG. 7 shows a western blot of cathepsin D (CatD), showing accumulation pre-pro-CatD in old versus young mouse hippocampal extracts. Pre-pro-CatD (ppCatD) requires autolytic processing by CatD in an acidic environment, so these data are consistent with impaired lysosome acidification in aging brain.

FIGS. 8A, 8B, 8C, 8D, and 8E show initial evidence of injury to neuronal fibers in proximity to p62-positive aggregates. FIG. 8A shows the hippocampus with YFP-expressing pyramidal neurons (green) shows denuded areas near p62 aggregates (14 mo-old mouse). High-power images from young (8B, 4 mo) versus old (8C, 24 mo) brain slices immunostained for p62 (red) and PV (green), allowing individual fibers to be better visualized. FIGS. 8D and 8E show higher magnification images showing fiber loss directly adjacent to p62-positive aggregates.

FIG. 9 shows lysosomes impairment in J774A.1 cells caused by treatment with pro-inflammatory LPS and the rescue of function by C3 co-treatment.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater gene expression, protein expression, amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller gene expression, protein expression, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treat,” “treating,” “treatment” and grammatical variations thereof as used herein, include partially or completely delaying, alleviating, mitigating or reducing the intensity of one or more attendant symptoms of a disease and/or alleviating, mitigating or impeding one or more causes of a disease. Treatments according to the invention may be applied preventively, prophylactically, palliatively or remedially. Prophylactic treatments are administered to a subject prior to onset (e.g., before obvious signs of disease), during early onset (e.g., upon initial signs and symptoms of disease), or after an established development of disease. Prophylactic administration can occur for several days to years prior to the manifestation of symptoms of an infection. In some instances, the terms “treat,” “treating,” “treatment” and grammatical variations thereof, include partially or completely rescuing lysosomal degredation and/or increasing initiation of autophagy as compared with prior to treatment of the subject or as compared with the incidence of such symptom in a general or study population. The reduction can be by 5%, 10%, 20%, 30%, 40% or more.

“Administration” to a subject includes any route of introducing or delivering to a subject the therapeutic agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. Administration includes self-administration and the administration by another.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Methods of Treating Neurodegenerative Disease

Age-related diseases are arguably the single greatest challenge for biomedicine in the 21st Century. The number of Americans over age 65 will be 70 million by 2030-nearly 25% of the population1. Current CDC data suggest that up to 30% of seniors over 65, and 50% of those over 85 will develop dementia. An ever-increasing number of these older adults will be facing chronic progressive neurodegenerative conditions such as Parkinson's disease, Alzheimer's disease, and other dementing disorders. Age, itself, is by far the single greatest common risk factor for all of these late-onset neurodegenerative diseases.

Alzheimer's disease (AD) is the leading cause of dementia, affecting 1 in 8 older Americans. While genetic forms of AD, which account for only 2% of patients, have strongly implicated a cascade of events, including β-amyloid (Aβ) accumulation (plaques), and deposition of abnormally phosphorylated tau (tangles) in AD, recent clinical trials targeting Aβ in sporadic AD patients have been disappointing. It is clear, however, that aging, itself, is the single greatest risk factor for the development of AD.

All major aging-related neurodegenerative diseases are characterized by proteinopathies, abnormal deposits of one or more proteins in brain (e.g. amyloid-β and tau in Alzheimer's disease (AD), α-synuclein in Lewy Body dementia, and TBP43 in frontotemptoral dementia/amyotrophic lateral sclerosis variant dementia, (the FTDs)). Although there are rare early-onset families with genetic forms of these diseases, the vast majority of patients develop sporadic, late-onset disease. Current CDC data indicate that up to 30% of seniors over 65, and 50% of those over 85 develop dementia; statistics which provide support for the observation that age, itself, is the single greatest risk factor for these late-onset neurodegenerative diseases (LO-NDs).

The brain has developed an elaborate system to degrade and remove excessive or damaged proteins. Proteins can be degraded intracellularly via the ubiquitin-proteasome system or the autophagy-lysosomal pathway (“self-eating”). Interestingly, one basic biological process that appears to decline with aging is autophagy, the highly choreographed and evolutionarily conserved process whereby eukaryotic cells deliver cytoplasmic components to lysosomes for degradation and recycling. Autophagy (specifically here macroautophagy) is activated in response to nutrient deprivation and other types of cellular stress to allow macromolecules to be recycled, and damaged organelles to be degraded and removed.

The autophagosomal/lysosomal system may also release its undigested contents to the extracellular space, where they can be degraded by proteases expressed and secreted by astrocytes, or taken up to be degraded intracellularly by glial cells, or exported into the blood or lymph. Impairment of any of these processes can lead to the formation of intracellular inclusions, extracellular aggregates, and potential neurodegeneration.

Another fundamental feature of aging is low-grade inflammation. Increasing evidence indicates that adverse health outcomes in older adults are strongly associated with the development of this proinflammatory state. Elevated levels of the inflammatory cytokine interleukin-6 (IL-6) in the aging brain are consistently associated with poorer cognitive function and sharper cognitive decline. Additionally, the IL-6 gene and the IL-6 receptor gene have been identified as AD risk genes. Circulating markers of inflammatory pathway activation, including IL-6, are also associated with enhanced risk of frailty, sarcopenia, disability, and early mortality in dozens of studies. In this context, it is shown herein that NADPH oxidase (Nox) is also increased in aging brains and that this up-regulation is dependent on IL-6. As reactive oxygen species (ROS) have been shown to impact protein clearance through proteasomes, autophagosomal/lysosomal systems and lysosomal exocytosis, it was asked whether hippocampal protein aggregates in aging brains contained p62 and additional proteins from autophagosomal/lysosomal systems, and whether their accumulation is due to defects in protein clearance as a result of Nox-derived ROS. Shown herein, age-related lysosome failure is due to IL-6 mediated activation of NADPH oxidase.

Inflammation in aging brain causes progressive failure of lysosomes which leads to accumulation of extracellular undigested contents. It is understood and herein contemplated that inhibition of any impairment of lysosomes or their function can significantly reduce the amount and size of intracellular inclusions, extracellular aggregates, and potential neurodegeneration. Because ROS production by Nox increased in aged brain and as ROS were known to impact protein clearance through proteasomal and autophagosomal/lysosomal systems, as well as lysosomal exocytosis, it is understood and herein contemplated that in one aspect, this inhibition of lysosomal impairment and ultimately treatment of an age-related neurodegenerative disease can comprise inhibiting at least one NOX isoform (such as, for example Nox1, Nox2, Nox3, Nox4, and/or Nox5) or a downstream Nox isoform effector (such as a reactive oxygen species (ROS) like superoxide or hydrogen peroxide. Accordingly, disclosed herein are methods of reducing age-dependent lysosome impairment and/or treating an age-related neurodegenerative disease, fibrotic disease, or rheumatological disease in a subject comprising administering to the subject a therapeutic agent; wherein the therapeutic agent inhibits nicotinamide adenine dinucleotide phosphate (NADPH)-oxidase (NOX) or downstream NOX effector.

As used herein, the disclosed methods can be used for the treatment or inhibition of any age-dependent lysosome impairment (such as, for example, lysosomal impairment present in hippocampal pyramidal neurons and parvalbumin interneurons) and/or age related neurodegenerative disease, fibrotic disease, or rheumatological disease of any preceding aspect, including, but not limited to neurodegenerative disease such as, for example, Alzheimer's disease, Parkinson's disease, Huntington's disease, spinocervellar ataxia type 1, age-related dementia, lewy body dementia, probably vascular dementia, frontotemporal dementia, and amyotrophic lateral sclerosis (ALS).

Because the impairment of lysosomes or lysosomal effector activity can occur not only after pathological symptoms of a disease have manifested, but days, weeks, moths, or even years prior to any pathological manifestation of a disease, it is contemplated that the above methods can be used not only therapeutically to reduce or treat and existing neurodegenerative disease, but also prophylactically to inhibit, delay, or reduce the magnitude and/or inhibit or delay the onset of clinical manifestation of disease. Accordingly, in one aspect, disclosed herein are methods of reducing age-dependent lysosome impairment and/or treating an age related neurodegenerative disease, fibrotic disease, or rheumatological disease in a subject comprising administering to the subject a therapeutic agent; wherein the therapeutic agent inhibits nicotinamide adenine dinucleotide phosphate (NADPH)-oxidase (NOX) or downstream NOX effector and wherein the therapeutic agent is administered at least one time at least 1, 2, 3, 4, 5, 6, 7 days, 2, 3, 4 weeks, 2, 3, 4, 5, 6, 7, 8, 9, 10, 11, 12 months, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more years prior to any clinical manifestation of disease. Also disclosed herein are methods of reducing age-dependent lysosome impairment and/or treating an age related neurodegenerative disease, fibrotic disease, or rheumatological disease in a subject comprising administering to the subject a therapeutic agent; wherein the therapeutic agent inhibits nicotinamide adenine dinucleotide phosphate (NADPH)-oxidase (NOX) or downstream NOX effector and wherein the therapeutic agent is administered at least once 1, 2, 3, 4, 5, 6, 7 days, 2, 3, 4 weeks, 2, 3, 4, 5, 6, 7, 8, 9, 10, 11, 12 months, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more years after to any clinical manifestation of disease.

It is understood and herein contemplated that the efficacy of the therapeutic agent can take multiple administrations to be effective. Accordingly, disclosed herein are methods of reducing age-dependent lysosome impairment and/or treating an age related neurodegenerative disease, fibrotic disease, or rheumatological disease in a subject comprising administering to the subject a therapeutic agent wherein the therapeutic agent inhibits nicotinamide adenine dinucleotide phosphate (NADPH)-oxidase (NOX) or downstream NOX effector and wherein the therapeutic agent is administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100 or more times. Administration can occur on a annual, semi-annual, quarterly, monthly, bi-weekly, weekly, daily, every 12 hours, every 8 hours, every 6 hours, every 5 hours, every 4 hours, every 3 hours, every 2 hours, every hour, or continuously as part of an automated delivery device.

The disclosed methods can use any small molecule, antibody, siRNA, antisense oligonucleotide, or peptide or any combination thereof capable of inhibiting the downstream effector or enzymatic activity of Nox or inhibiting a Nox isoform from assembling or carrying out its enzymatic activity. For example, in one aspect, the agent can be a small molecule comprising diphenylene iodonium. Apocynin, 2-(2-chlorophenyl)-4-[3-(dimethylamino)phenyl]-5-methyl-1H-pyrazolo[4,3-c]pyridine-3,6-dione (GKT-831), Rapamycin, dismutazyme, a malonic acid derivative of a carboxyfullerene (such as, for example C60), an acetic acid derivative of a carboxyfullerene (such as, for example C60), or any combination thereof. Thus, in one aspect, disclosed herein are methods of reducing age-dependent lysosome impairment and/or treating an age related neurodegenerative disease, fibrotic disease, or rheumatological disease in a subject comprising administering to the subject a therapeutic agent; wherein the therapeutic agent inhibits nicotinamide adenine dinucleotide phosphate (NADPH)-oxidase (NOX) or downstream NOX effector and wherein the therapeutic agent; wherein the therapeutic agent comprises diphenylene iodonium. Apocynin, 2-(2-chlorophenyl)-4-[3-(dimethylamino)phenyl]-5-methyl-1H-pyrazolo[4,3-c]pyridine-3,6-dione (GKT-831), Rapamycin, dismutazyme, a malonic acid derivative of a carboxyfullerene (such as, for example C60), an acetic acid derivative of a carboxyfullerene (such as, for example C60), or any combination thereof.

One of the primary effectors of Nox that has a detrimental effect on lysosomes is reactive oxygen species (ROS) and it is further understood and herein contemplated that several of the Nox inhibitors (for example dismutazyme, a malonic acid derivative of a carboxyfullerene (such as, for example C60), an acetic acid derivative of a carboxyfullerene (such as, for example C60), and/or any combination thereof) are effective because they inhibit ROS as a downstream Nox effector. However, such inhibition is not merely limited to Nox initiated ROS, but any ROS regardless of source. This inhibition, reduction, and/or elimination of ROS can improve lysosome function. Accordingly, contemplated herein are methods of improving lysosome function in a subject comprising administering to the subject a therapeutic agent (such as, for example, dismutazyme, a malonic acid derivative of a carboxyfullerene (such as, for example C60), an acetic acid derivative of a carboxyfullerene (such as, for example C60), and/or any combination thereof); wherein the therapeutic agent reduces, inhibits, and/or eliminates reactive oxygen species.

In one aspect, it is understood and herein contemplated that IL-6 is increased in the brain of aged humans, mice, rats, and monkeys. Moreover, the increase in IL-6 is both necessary and sufficient for the induction of Nox. Accordingly, one manner in which the enzymatic activity of or assembly of Nox can be inhibited is to prevent its induction. Thus, in one aspect, disclosed herein are of reducing age-dependent lysosome impairment, improving lysosome function, increasing initiation of autophagy, and/or treating an age related neurodegenerative disease, fibrotic disease, or rheumatological disease in a subject comprising administering to the subject a therapeutic agent; wherein the therapeutic agent inhibits Nox assembly and/or enzymatic activity, and wherein the therapeutic agent decreases the amount of IL-6 present in hippocampal pyramidal neurons and parvalbumin interneurons, inhibits the increase of IL-6 in hippocampal pyramidal neurons and parvalbumin interneurons, or inhibits the induction of Nox by IL-6 (i.e., reduces IL-6 mediated NOX activation, thereby causing NOX inhibition). For example, the therapeutic agent can be an anti-IL-6 antibody or small molecule. For example, the IL-6 antibody can be tocilizumab, sarilumab, olokizumab, elsilimommab, CPSI-2364, galiellaclactone, and/or sirukumab.

In one aspect, it is understood and herein contemplated that IL-6 activates NOX via though the activation of STAT3. Thus, in one aspect, disclosed herein are methods of reducing age-dependent lysosome impairment, improving lysosome function, increasing initiation of autophagy, and/or treating an age related neurodegenerative disease, fibrotic disease, or rheumatological disease of any preceding aspect wherein the therapeutic agent is a signal transducer and activator of transcription 3 (STAT3) inhibitor, and wherein the therapeutic agent reduces STAT3 mediated NOX activation, thereby causing NOX inhibition. For example, the therapeutic agent can be an anti-STAT3 antibody or small molecule STAT3 inhibitor such as, for example, niclosamide, STAT3 inhibitor VI (S31-201), STA-21, WP1066, Cucurbitacin I, curcumin, auranofin, and/or nifuroxazide.

Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with a downstream NOX effector (such as, for example superoxide, hydrogen peroxide, or other reactive oxygen species) or a NOX isoform (such as, for example NOX1, NOX2, NOX3, NOX4, or NOX5) such that NOX is inhibited from assembling or carrying out its enzymatic activity. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain inhibition of downstream NOX effector activity (such as, for example superoxide, hydrogen peroxide, or other reactive oxygen species) or a NOX isoform (such as, for example NOX1, NOX2, NOX3, NOX4, or NOX5) such that NOX is inhibited from assembling or carrying out its enzymatic activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### 2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### B. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Age-Dependent Accumulation of Protein Aggregates in Mouse Hippocampus is Reduced by NADPH Oxidase Inhibition

The brain has developed an elaborate system to degrade and remove excessive or damaged proteins. Proteins can be degraded intracellularly via the ubiquitin-proteasome system or the autophagy-lysosomal pathway. The autophagosomal/lysosomal system can also release its undigested contents to the extracellular space, where they can be degraded by proteases expressed and secreted by astrocytes, or taken up to be degraded intracellularly by glial cells, or exported into the blood or lymph. Impairment of any of these processes can lead to the formation of intracellular inclusions, extracellular plaques, and potential neurodegenerative processes.

Convergent evidence suggests that protein clearance, particularly degradation through lysosomes, becomes progressively less efficient in both aging and neurodegenerative diseases. Genome-wide association studies (GWAS) have identified lysosomal genes (including CLU, BIN1, and PICALM) as Alzheimer's disease (AD) risk genes, and a number of other lysosome-associated genes were identified as risk genes for Parkinson's disease (PD). Studies on brain-derived exosomes, secreted membrane-delimited vesicles, found increased levels of lysosomal proteins (Lamp2 and ubiquitin) in patients up to ten years before the onset of AD. These results suggest that lysosomal dysfunction could occur in the aging brain years before the onset of the pathological changes characteristic of these neurodegenerative diseases. The decline in protein clearance also appears to occur during normal aging as inferred from observations of intracellular lipofuscin and extracellular protein aggregates in brains of normal old animals across species. Extracellular protein aggregates were first identified as periodic acid-schiff-positive granules and were subsequently shown to originate from both neurons and glial cells. Among the detected proteins in these aggregates is p62/sequestosome-1, a multifunctional protein involved in protein trafficking and autophagy. p62 can bind and sequester ubiquitinated proteins, which can be released later for proteasomal degradation or alternatively degraded through the autophagy pathway. The presence of p62 in the protein aggregates implies that old mouse hippocampus does not efficiently eliminate the cargo of autophagosomes, most likely due to a decline in lysosomal protein degradation. Such age-dependent decline in protein clearance can be a common theme for normal aging and neurodegenerative diseases.

Another fundamental feature of aging is low-grade inflammation. Increasing evidence indicates that adverse health outcomes in older adults are strongly associated with the development of this proinflammatory state. Elevated levels of the inflammatory cytokine interleukin-6 (IL-6) are consistently associated with poorer cognitive function and sharper cognitive decline. Additionally, the IL-6 gene and the IL-6 receptor gene have been identified as AD risk genes. Circulating markers of inflammatory pathway activation, including IL-6, are also associated with enhanced risk of frailty, sarcopenia, disability, and early mortality in dozens of studies. In this context, it is shown herein that NADPH oxidase (Nox) is also increased in aging brains and that this up-regulation is dependent on IL-6. As reactive oxygen species (ROS) have been shown to impact protein clearance through proteasomes, autophagosomal/lysosomal systems and lysosomal exocytosis, it was asked whether hippocampal protein aggregates in aging brains contained p62 and additional proteins from autophagosomal/lysosomal systems, and whether their accumulation is due to defects in protein clearance as a result of Nox-derived ROS.

a) Methods

### (1) Materials

The following antibodies were used: rabbit anti-parvalbumin (Swant, PV-25), mouse monoclonal antiparvalbumin (Swant, PV-235), rabbit anti-calretinin (Swant, 7699/4), rabbit anti-p62 (Sigma, P0067), rabbit anti-LC3B (Sigma, L7543), mouse mab anti-parkin (Abcam, ab77924), rabbit anti-pinkl (Abcam, ab 23707), rabbit mab anti-lamp2b (Abcam, ab125068), mouse mab anti-cytochrome P450 (EMD Millipore, mab10037), mouse mab anti-reelin (EMD Millipore, mab 5364), mouse mab anti-GAD67 (EMD Millipore, mab5406), rabbit anti-α-synuclein (EMD Millipore, ab5038), rabbit anti-Iba (Wako, 019-19741), and rat mab anti-GFAP (Calbiochem, 345860). VECTASHIELD and VECTASHIELD with DAPI mounting medium were from Vector Labs (Burlingame, Calif.). Tyramide Signal Amplification (TSA) Plus Fluorescein and cyanine-3 Kits were from PerkinElmer (Waltham, Mass.).

### (2) Mice

Animal studies were approved by the Animal Care Program at the University of California, San Diego, and Vanderbilt University Medical Center and are in accordance with the PHS Guide for the Care and Use of Laboratory Animals, USDA Regulations, and the AVMA Panel on Euthanasia. Young (3 to 5 month) and old (17 to 27 month) C57BL/6 mice, Tam-Thy1-YFP-Cre, tdTomato or 24 month old GFAP/GFP transgenic mice (JAX, 010835) were used for experiments. Lines which have the Tam-Thy1-YFP-Cre express both the Cre recombinase and YFP under control of the Thy1 promoter, which drives expression in a majority of hippocampal and cortical excitatory neurons, but not inhibitory neurons. Tam-Thy1-YFP-Cre:Stat3flox/flox (TF) mice have been generated, and have also been crossed with the Rosa26-tdTomato reporter mouse line to produce Tam-Thy1-YFP-Cre:Stat3flox/flox:tdTom (TFTD) mice. These reporter mice will express tdTom fluorescence in response to tamoxifen. Mice were deeply anesthetized with isoflurane before they were perfused transcardially with ice-cold phosphate buffered saline (PBS). After the PBS flush, mice were further perfused with a 4% PFA solution in 0.01M PBS for 10 minutes. Whole brains were removed and post-fixed in 4% PFA at 4° C. overnight and then sliced using a Vibratome into 50 μm sections. The sections were kept in 30% sucrose, 30% ethylene glycol, and 1% PVP-40 at −20° C. until they were ready to be processed for immunostaining.

### (3) Treatment with Apocynin

Mice were treated with apocynin in drinking water (initial dose 5 mg/kg and then ramping up to 100 mg/kg) beginning at 12 months old for 5 months. Mice were then perfused with PBS followed by PFA and brains were harvested at seventeen months old.

### (4) Immunofluorescence

An antigen retrieval method was performed on the sections for most antigens by heating in 10 mM citrate buffer (pH 6.0) for 10 minutes at 90° C. Sections were then treated with 3% H₂O₂ and 10% ethanol in PBS for 15 minutes to inactivate endogenous peroxidase activities and then blocked in 2% bovine serum albumin (BSA) and 0.3% Triton X-100 in PBS at room temperature for 1 hour, followed by incubation with primary antibodies (diluted at 1:500 to 1:6000 for detection by fluorescent secondary antibodies, or at 1:5000 to 100,000 for TSA detection) in blocking buffer for periods ranging from overnight to 72 hours at 4° C. Sections were then washed and stained with Alexa Fluor 488 or 568 conjugated secondary antibodies (1 μg/ml) or HRP-conjugated secondary antibodies (1:3000) in 2% BSA and 0.3% Triton X-100 in PBS for 2 hours at room temperature. With HRP-conjugated secondary antibodies, fluorescence was developed with fluorescein or cyanine 3 TSA amplification systems. For double fluorescent staining of mouse brain sections using primary antibodies from the same species, highly diluted antibodies were used for detection of one antigen with TSA, followed by detection of the second antigen, usually p62, with regular fluorescent immunostaining Sections were washed and mounted on clean microscopy slides with VECTASHIELD mounting medium with DAPI.

### (5) Microscopy and Image Analysis

Images were captured using Zeiss LSM 510 or LSM 810 multiphoton laser confocal microscopes. For thioflavin S, excitation was achieved with the 458 nm laser line and fluorescence was detected between 480-520 nm. For Alexa Fluor 488 and fluorescein TSA, excitation was achieved with the 488 nm laser line, and fluorescence between 500-550 nm was detected. For Alexa Fluor 568 or cyanine 3 TSA, excitation was achieved with the 543 nm laser line, and fluorescence with wavelengths longer than 560 nm were detected. For lipofuscin, excitation was achieved with the 458 nm laser line, and fluorescence between 571-606 nm was detected. Confocal microscope settings were kept constant for each experiment. Images were then analyzed and processed using Metamorph or Fiji software. To quantify the clusters of p62-containing aggregates, images were taken for 6 hippocampal regions from three sections per mouse, with 4 mice for each group. Hippocampal regions were divided into different subregions, and p62 clusters were counted. After thresholding the pictures, clusters were individually circled and effective areas were measured using Fiji. Areas of clusters of aggregates in each subregion were then summed. Areas of aggregates and counts of clusters from the highlighted subregion (FIG. 5A) were presented because aggregates were mostly formed in this subregion in less advanced aged mice. The analyzer was blinded to samples.

### (6) Immunoblots

Tissue lysates were prepared from flash frozen hippocampal tissues. Tissues were thawed and homogenized in cold Super RIPA buffer (10 mM phosphate buffer, pH 7.4, 150 mM NaCl, 1 mM EGTA, 1% Ipegal-630, 1% deoxycholic acid, 0.5% SDS, 1 mM molybdic acid, and 1 mM DTT) with Protease inhibitor cocktail (Roche, 04 493124001). Tissue lysates were cleared by centrifugation at 12000 RPM for 10 minutes. Protein concentrations were determined by the BCA Protein Assay kit (Thermo Scientific, 23225). 20 to 40 μg of proteins were subjected to SDS-PAGE, transferred to PVDF membranes, blocked with 5% non-fat milk in 0.1% TBS-Tween for 1 hour at room temperature, and probed with primary antibodies overnight at 4° C. Following primary antibody incubation, the blots were probed with horseradish peroxidase-conjugated secondary antibody for one hour at room temperature, and visualized with Pierce ECL Western blotting substrate (32106).

### (7) Statistical Analysis

All statistics were performed using SigmaPlot 11.0.

b) Results
(1) Clustered Protein Aggregates in the Hippocampi of Aged Mice

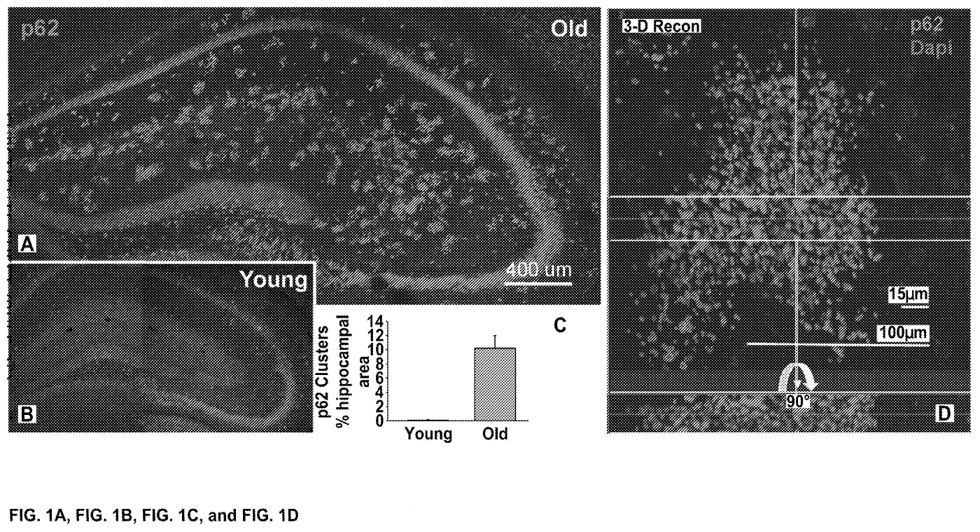

As shown below, aging mice demonstrate large (100-300 μm) clusters/aggregates of deposited lysosomal proteins, including p62 (FIG. 1). These aggregates were observed throughout the hippocampus, and in old animals covered a significant percentage of that structure. Brain accumulation of p62 aggregates has previously been reported in old rats, humans, and in invertebrates (i.e. in Drosophila, the p62 ortholog, Ref(2)P). To visualize autophagy in mouse brains, brain sections were stained with antibodies against p62, a protein involved in autophagy and enriched in autophagosomes. p62 fluorescence was observed in pyramidal neurons and local interneurons in hippocampus, with higher intensity in the latter. At high magnifications, punctate fluorescence was observed in the soma of pyramidal neurons and interneurons in both young and old mice. These puncta were distinct from lipofuscin bodies that are pronounced in aged mice. The global level of p62 as well as that of LC3BII, the lipidated LC3B form, were slightly increased in old mouse hippocampi. In addition, the level of LC3BII was low compared with that of intact LC3BI in both young and old mouse hippocampal extracts. Taken together, while the global level of autophagosomes in aged brains was not significantly increased, there can be a preferential increase in autophagosomes in parvalbumin neurons during aging.

The distinct difference in p62 staining between old and young mice was the clustered granular aggregates observed in old but not young mice (FIGS. 1A, B and C). It was also seen in mice of one year old mice. This particular pattern was observed in hippocampus and piriform cortex. High resolution imaging (FIG. 1D) indicated these aggregates were made up of small 3-8 μm deposits. Because p62 is preferentially degraded by lysosomes, and accumulates rapidly when lysosome proteolytic activity is impaired, this indicated that lysosomes were not efficiently degrading contents, and that other protein “cargo” canalso have escaped degradation.

(2) Additional autophagy-relevant proteins in p62 positive aggregates

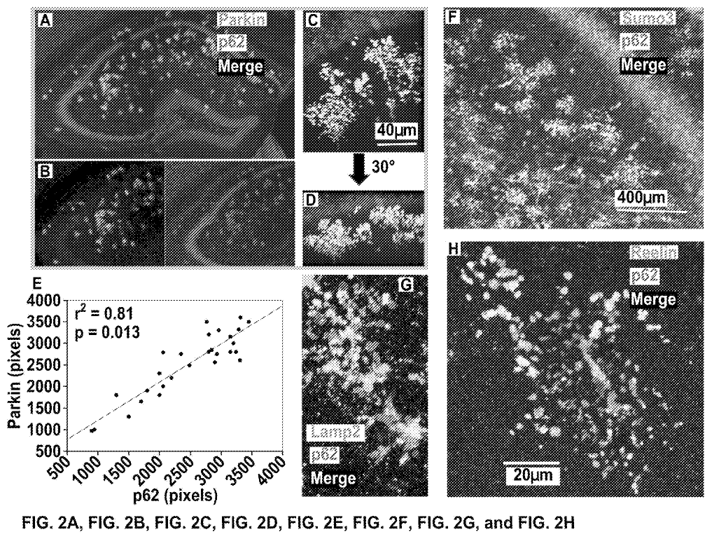

The possible origins of these aggregates were further examined by immunostaining. p62 and Parkin, a protein involved in mitophagy and PD. Parkin was essentially in all p62 positive aggregates (FIGS. 2A, 2B, 2C, 2D, and 2B). A number of other proteins, including reelin, parvalbumin (PV), pinkl, p450, calretinin, and α-synuclein also were found in the aggregates. This is consistent with a generalized defect lysosomal degradation of cargo proteins. To quantify the degree of colocalization of proteins within the aggregates, the Imaris CoLoc image analysis software available was used along with Pearson's correlation coefficient (PCC). Calculations for p62 and parkin are shown in FIGS. 2F and 2G.

(3) Cellular Origins of Protein Aggregates in Mouse Hippocampus

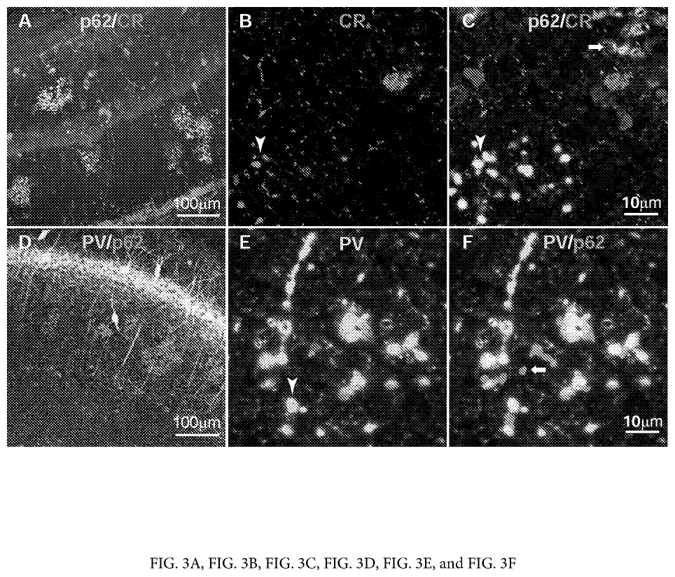

Studies showed that reelin was contained in similar protein aggregates. As reelin was synthesized and secreted from calretinin neurons, it was asked whether p62 containing aggregates were also associated with calretinin neurons. While most of the p62 positive aggregates did not contain calretinin, some p62 positive aggregates were also positive for calretinin (FIG. 3A-C). Thus, calretinin neurons also contributed to p62 positive protein aggregates. As p62 vesicles were most pronounced in parvalbumin neurons, a possible indication of impairment in clearance of autophagosomal contents, it was asked whether they contributed to the formation of protein aggregates. Most p62 positive aggregates did not contain parvalbumin. However, some p62 positive aggregates were observed in parvalbumin positive fibers (FIG. 3D-F). In addition, p62 positive parvalbumin fibers appeared to be rounder and more swollen (roundness=0.7069±0.121 (mean±standard deviation), n=23), p<0.001) compared with p62 negative parvalbumin fibers roundness=0.44536±0.1802, n=18), which were not different from parvalbumin positive fibers in young mice (roundness=0.5306±0.2023, n=25). Thus, both calretinin and parvalbumin neurons can contribute to the formation of protein aggregates.

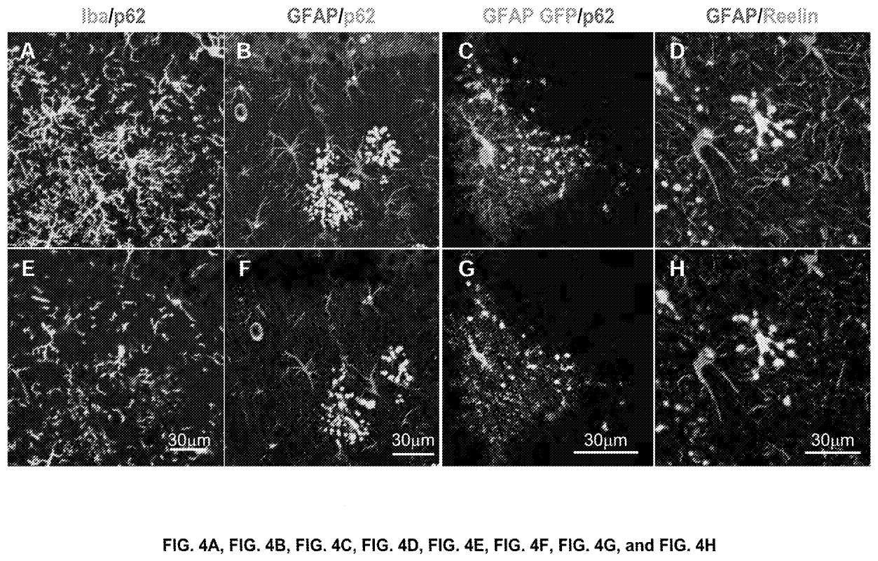

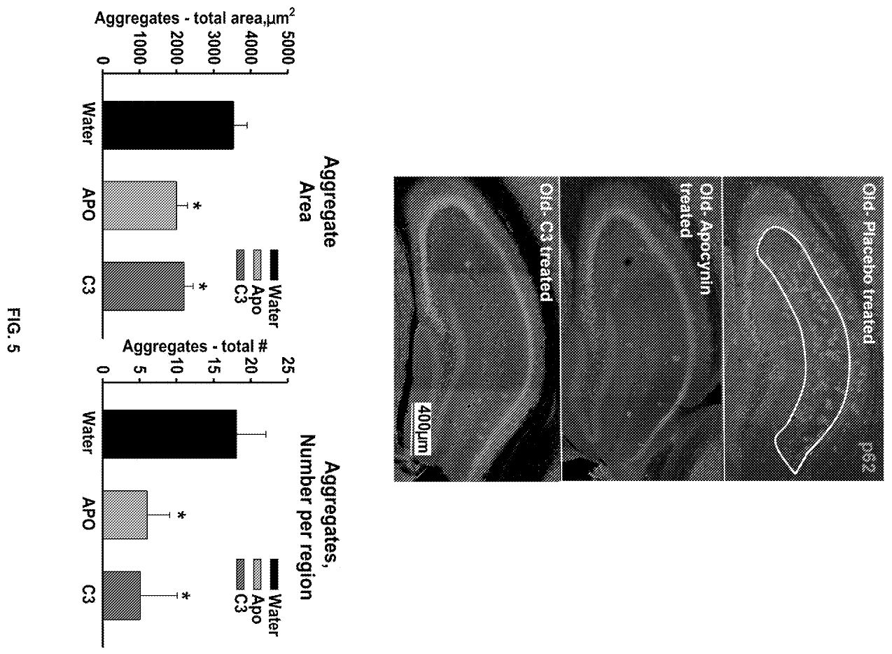

Several groups showed that these protein aggregates were both extracellular and intracellular, and about 60% were associated with astrocytes (Jucker et al., 1994; Kuo et al., 1996). While no significant p62 positive aggregates were observed in Iba positive microglia, smaller p62 puncta can be observed inside some GFAP labeled astrocytes (FIGS. 4A-B and E-F). In addition, some p62 positive aggregates appeared to cluster around astrocytes. As GFAP did not label all fine projections of astrocytes, p62 staining in brains of mice was examined with GFP expression driven by the GFAP promoter (GFAP-GFP mice). p62 was frequently observed in association with GFP labeled astrocytes in GFAP-GFP mice (FIGS. 4C and G). The majority of p62 positive clusters were not associated with GFP labeled astrocytes, but as not every astrocyte expressed GFP in these mice, the association between protein aggregates and astrocytes can be higher than it appeared here. Agreeing with earlier observations, reelin positive aggregates were also observed to be associated with, and inside, astrocytes (FIGS. 4D and H). These observations were consistent with the proposition that protein aggregates derived from neurons can be taken up by astrocytes.

(4) Evidence that Activation of Nox is Involved in Impaired Processing of Lysosome Cargo.

As discussed above, increased Nox2 expression and activity in brain of aging WT mice. Nox is a multimeric enzyme complex first described as the respiratory burst oxidase in neutrophils. The Nox family of oxidases can produce large amounts of predominantly superoxide, the one electrode reduction product of molecular oxygen, for both pathogen killing, but also for intracellular signaling through a variety of redox mechanisms. The Nox family of proteins are widely expressed, with Nox1, 2, and 4 expressed in rodent brain, and all of these, plus Nox 5, expressed in human brain. Nox 5, which calcium-dependent, is not present in rodents, however. Nox expression can be increased in by a variety of stressors and injury conditions. IL-6 and signal transducer and activator of transcription 3 (Stat3) are involved in activation of Nox2. Nox2, Nox4, and the regulatory subunit, p22phox, are induced in aging brain, and showed that Nox2 activity was highly upregulated in normally aging wild-type mice.

Here is elucidated whether Nox activity contributes to lysosomal dysfunction by treating mice long-term with the Nox inhibitor, apocynin (50 mg/kg/day), or a superoxide dismutase mimetic (C3, 1 mg/kg/day) and found a significant improvement in lysosome function, as demonstrated by a reduction of both the number, and size of p62-positive aggregates (FIG. 5) (and parkin-positive aggregates. FIG. 5 shows data from females, but a second cohort of aged male mice showed similar effects, indicating that Nox impairs clearance of proteins in both sexes. These data are significant because they directly link Nox activity with defects in lysosome clearance of material. The ability to modify lysosomal function and clearance of cargo in a non-genetic manner can allow studies on the mechanisms of age-related lysosome dysfunction throughout the lifespan.

(5) Enhancing Delivery of Cellular Material for Degradation by Impaired Lysosomes can Worsen Lysosome Function.

Overburdening failing lysosomes can produce tissue injury. They found that despite increased autophagosome formation and enhanced lysosome biogenesis, there was a progressive accumulation of LC3b-II, SWQTM1/p62, and large engorged lysosomes in brain. The authors concluded that sustained upregulation of autophagy in the face of declining lysosomal clearance of substrates not only results in increasingly defective lysosomes, but in fact contributed to neuronal injury. Drugs such as rapamycin, which activate the initial steps in autophagy to promote delivery of damaged macromolecules/organelles to lysosomes, have been beneficial in a number of diseases models, but there has been recent appreciation of the fact that increasing the load of material to dysfunctional lysosomes can worsen autophagy, rather than enhance it. In fact, a recent paper using rapamycin in an AD mouse model showed some behavioral improvement with treatment, but no difference in clearance of plaques or tangles, indicating that rapamycin was unable to overcome impaired lysosome proteolysis. These results indicate that to gain the full benefits of enhanced autophagy by rapamycin or rapalogs, normalizing lysosome function first can be important.

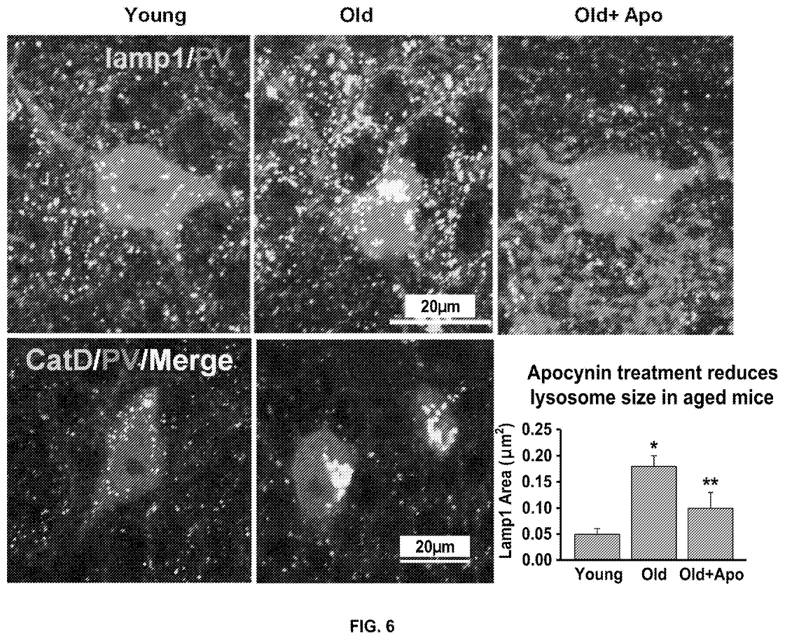

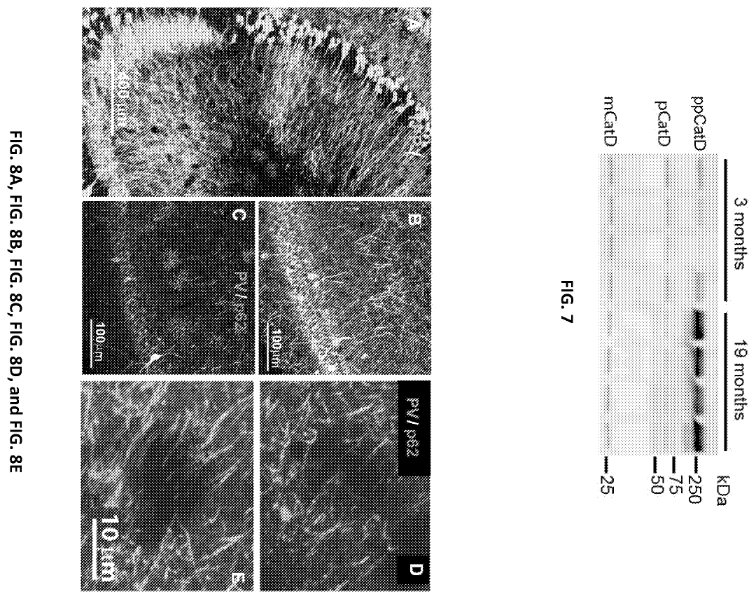

In this context, it was asked whether ineffective lysosome degradation of contents could be seen as enlarged, (presumably engorged) lysosomes (FIG. 6) in old mice. Such a finding is consistent with the proposed impaired acidification of lysosomes, as it is known that direct inhibition of lysosome acidification by the lysosomal v-ATPase inhibitor bafilomycin rapidly increases lysosome size within hours, indicating that lysosome size is a dynamic index of effective lysosome function. As shown (FIG. 6), both the average and cumulative sizes of lamp1-positive lysosomes were significantly larger in neurons of old animals Nox inhibition produced significant normalization (reduction) of intracellular lysosome size in neurons using both lamp1 (FIG. 6) and CatD immunostaining in aged mice treated with apocynin compared to water. Based on these results, p62 aggregate size, numbers, and % of hippocampal area occupied were used as measures of static long-term lysosome dysfunction, and to use neuronal lysosome size and numbers (using lamp1 and CatD as markers) as measures of dynamic changes in lysosome activity.

c) Discussion

Protein aggregates in aged hippocampi have been detected with antibodies against a variety of antigens, but the significance of these aggregates is not clear. The study shows that autophagosomal/lysosomal cargo proteins such as p62, Parkin, Pinkl, and Lamp2B are also included in these aggregates. These observations indicate that age-dependent accumulations of protein aggregates can result from the inability of hippocampal tissues to efficiently remove or degrade proteins from the autophagy-lysosomal pathway. In addition, parvalbumin or calretinin GABAergic neurons, as well as astrocytes, can contribute to the formation of these aggregates. The accumulation of protein aggregates during aging is attenuated by Nox inhibition, which shows that Nox derived ROS can impact protein clearance from mouse hippocampus.

(1) Possible Deficit in the Autophagy-Lysosomal Pathway in the Hippocampus of Aged Mice

Autophagy is predominantly cytoprotective, allowing macromolecules to be recycled, and damaged organelles to be degraded and removed. Impairment of autophagy induces inflammation and shortens lifespan, while activation of autophagy reduces disease pathology in multiple disease models and increases lifespan. It is believed that autophagy is impaired during aging, but both increases and decreases in autophagic flux in aged animals have been reported. What seems to be more consistent is a decline in lysosomal function in senescent cells. By immunostaining with antibodies against LC3B and p62, it was observed some puncta resembling autophagosomes, but there was no significant global increase in aged hippocampi. This is in agreement with results from Western blot experiments demonstrating that the amount of LC3BII is insignificant compared with that of intact LC3BI. However, the slight increase in both p62 and LC3BII in old mice showed by Western blot can be interpreted as a result of the declined capacity of the autophagy-lysosomal pathway. Finally, vesicular like p62 imunoreactivities are larger and more abundant in parvalbumin interneurons than those in pyramidal neurons, indicating that autophagy or lysosomes can be preferentially impaired in these cells.

(2) Protein Aggregates in Aged Mice Contain Proteins from the Autophagy-Lysosomal Pathway and are of Neuronal and Glial Origins.

The protein aggregates detected with p62 immunostaining are similar to the periodic acid-schiff positive granules. These granules contain heparin sulfate proteoglycans and laminin, as well as reelin, and have been proposed to originate from both neurons and glial cells. It was demonstrated herein that these aggregates contain Parkin, Pinkl, and Lamp2B, as well as cytochrome P450, indicating that protein degradation can be compromised in aged mice following autophagy or mitophagy. These aggregates also appear to originate from both neuronal and glial cells. Some p62 positive aggregates are associated with astrocytes, as revealed by experiments with GFAP-GFP mice. Some p62 positive aggregates are also observed in parvalbumin or calretinin containing fibers. If these markers label similar protein aggregates, then granules associated with astrocytes also contain reelin secreted from neurons, which is confirmed by the double immunostainings for GFAP and reelin. These observations also imply that astrocytes are able to take up proteins of neuronal origin. This traffic of neuronal components to astrocytes is not uncommon. For example, astrocytes have been shown to mediate the removal of AP deposits and synaptic components. As astrocytes tend to have their own territories, the accumulation of these aggregates clustered around a particular astrocyte can reflect its decreased ability to uptake or degrade extracellular protein aggregates. Prolonged exposure of these protein aggregates to the extracellular space can lead to further modifications by ROS or extracellular enzymes, making them more resistant to subsequent degradation and removal.

(3) Nox-Derived ROS and Protein Clearance

Treatment of middle age mice with the Nox inhibitor apocynin for five months reduces hippocampal aggregates, which consistent with the understanding that Nox derived ROS impairs protein clearance from hippocampus. ROS are known to have complex effects on the proteasomal system. While oxidized proteins are better substrates for this system, and acute ROS exposure can decrease or increase protein degradation through this system, the effects of chronic ROS exposure on protein degradation occurring during aging have not been reported to the knowledge. Similarly, ROS also affect initiation of autophagy and the subsequent degradation of autophagic cargos in lysosomes. Specifically, enzymes involved in lysosomal protein degradation such as cathespsin B and L have active cysteine residues that can be inactivated by ROS. Further, the assembly and activity of V-ATPases on autophagosomes and lysosomes are also regulated by ROS, leading to the alkalinization of these organelles. This results in the inefficient digestion of lysosomal cargos. Finally, ROS can also promote lysosomal exocytosis, releasing undigested proteins from lysosomes. All of these can contribute to the formation of protein aggregates in the hippocampus of aged brain. In summary, the results presented here show that protein aggregates are formed through extrusion of undigested proteins from aged parvalbumin and calretinin interneurons, in a response to impaired protein degradation by autophagosomal/lysosomal systems resulting from elevated levels of ROS derived from Nox.

Example 2: Current Anti-Inflammatory Drugs do not Target the Appropriate Pathways

To address why identifying the mechanisms responsible for age-related lysosome clearance of cargo is critically important to developing new therapeutic approaches, it is important to note the following. There are no drugs which activate lysosomes directly. Commonly used non-steroidal anti-inflammatory drugs do not reduce IL-6 expression, and in certain situations, can actually increase activation of the IL-6/Stat3 cascade. Thus, new drugs are needed to treat inflammation involving this pathway, as well as downstream mediators, for example NADPH oxidase.

There are now three clinically approved anti-IL-6 immunotherapies approved specifically for rheumatoid arthritis that is refractory to other treatments. These can be attractive as an intervention. However, these immunotherapies are directed against IL-6 membrane receptors (IL-6R), and as much of IL-6 signaling goes through soluble receptors which bind to gp130, not the IL-6R, much of IL-6 signaling is not targeted by these immunotherapies. In addition, both formulations have proven to have adverse reactions especially with sustained treatments, none cross the blood-brain barrier, and these have only been used in individuals with refractory rheumatoid arthritis, not long-term in healthy older patients. Development of small molecule Nox inhibitors is an active area of research for a number of pharmaceutical companies, with one or more in clinical trials for a range of applications. However, each inhibitor targets a specific Nox isoform, and more than one isoform can be induced by inflammatory cytokines. Finally, there has been little emphasis to date on Nox5, which can play a key role in the CNS due to its calcium dependence.

Example 3: Lysosomal Dysfunction During Normal Aging is Due to Inflammatory Activation of NADPH Oxidase Via Increased IL-6 and/or Stat3 Pathway Activation.

Provided herein is exciting new data supporting the first step in this link by demonstrating that mice treated with the NADPH oxidase (Nox) inhibitor, apocynin, have a robust and highly significant reduction in undegraded lysosome material throughout brain (FIG. 5). These are the first data showing that inhibiting Nox activity can improve lysosome function in brain in vivo. Analyses also indicated that apocynin significantly reduced the size of lysosomes, which are enlarged in aged animals (FIG. 6), back towards the size of those found in young animals. Based on the observation herein that IL-6 is responsible for Nox2 induction in aging brain, and thus here, it is determined whether IL-6 mediates lysosome dysfunction. In addition, because IL-6 regulates Stat3, and the promoter regions for Nox subunits possess GAS (Stat3 target) sequences, it is likely that Stat3 is involved in Nox-mediated lysosome deficits. IL-6 knockout mice, which have a targeted deletion of Stat3 in neurons, and pharmacological inhibition of Nox-derived superoxide production can be used to test each of these possibilities. In support of this, 4 month and 24 month-old C57Bl/6 mice injected with IL-6 (5 μg/kg) on two consecutive days had induction of Nox2, Nox4 and p22phox, as measured by qRT-PCR, Western blot, and immunostaining of brain slices. This increase occurred not only in microglia, but cortical and hippocampal neurons, and in synaptosomes. To confirm anti-Nox2 Ab specificity, slices and tissue from gp91_{phox-/-} (Nox2_{-/-}) mice were always included as controls.

a) Mouse In Vivo Studies.

To test the understanding that Nox-derived superoxide leads to dysfunctional lysosomes in aged brains, cohorts of mice are treated with drugs which test aspects of Nox effects on lysosome function. These drugs and the rationales are included here; 1) Nox inhibitor apocynin (100 mg/kg/day, as in FIG. 5, improved cargo clearance), 2) the superoxide dismutase mimetic (C3, 1 mg/kg/day) to reduce tissue levels of superoxide, and 3)N-acetyl cysteine (10 mg/kg/day) to increase cellular levels of glutathione and reverse thiol oxidation of cathepsins. All drugs are given in the drinking water, and the control groups (two) receive regular drinking water as the control. Mice start treatments or control at 3 ages; 5 mo. (young adult), 12 mo. (early aggregate deposition observed), and 18 mo. (old), with 5 males and 5 females at each age, and all can be sacrificed at 22 mos. of age (when significant deposits are seen along with age-related cognitive impairment). Thus 30 mice are enrolled for each treatment group for imaging studies. Outcome measures include p62 cluster size, number, and percent of hippocampal area (FIG. 5), intra-neuronal lysosome size using lamp1 (FIG. 6), and evidence of other proteins co-localized with aggregates (e.g. FIG. 2). The start of treatments can be staggered so that all three age groups are 22 months of age at sacrifice, so they can be processed concurrently. To allow protein expression and cathepsin activity to be analyzed, a second similar set of treatment groups are needed, and initiation of treatments can be staggered as above. Thus, additional mice are needed for protein and cathepsin activity analyses.

b) Immunostaining Procedures with Mouse Brain Slices.

Mice can be anesthetized with isoflurane, and transcardially perfused with ice-cold phosphate buffered saline (PBS) for 1 minute, followed by perfusion with cold 4% paraformaldehyde (PFA) in 10 μM PBS for 5 minutes. Whole brains are removed and post-fixed in 4% PFA at 4° C. overnight, switched to 2% PFA for an additional 24 hours, then sliced on a Vibratome to generate 50 μm sections which are maintained in 30% sucrose, 30% ethylene glycol, 1% PVP-40 at −20° C. until they are ready to be processed for immunostaining. Floating sections are then blocked, immunostained with primary antibody, washed, labelled with fluorescent secondary antibody, and mounted in Vectashield mounting medium on glass slides for imaging. All analysis is performed by individuals blind to treatment information. Antibodies to be used include (Vendor, CAT #, Dilution): rabbit anti-p62 (Sigma, P0067, 1:4K), mouse mab anti-parkin (Abcam, ab77924, 1:1K), rabbit anti-pinkl (Abcam, ab23707, 1:5K with TSA), rabbit mab anti-lamp2b (Abcam, ab125068, 1:5K with TSA), and rabbit anti-LC3B (Sigma, L7543, 1:1 to 2K) Immunostained sections are imaged on a Zeiss LSM 880 2-photon confocal system. Slides are stored in the dark when not being imaged Immunofluorescence for a given autophagy marker can be quantified by the Zeiss Zen Blue analytical software or Image J. Lysosome failure can be defined as an increase in p62, and lamp2 deposits in the presence of increased levels of LC3b-II, all by immunostaining.

c) Nox Isoform Changes in Aging and Inflammatory Brains.

An increase in Nox2 and Nox4 expression in old brain, observed in microglia, but also extensively in neurons. In these experiments, the role of Nox2 and Nox4 changes in brains of aging mice can be determined. Nox expression levels can be initially quantified by qPCR and regular western blotting, and their distribution can be examined by co-immunostaining with specific neuronal, glial, endosomal and lysosomal markers. CNS inflammation can be examined by quantifying inflammatory markers including TNFα, IL1β, IL6, Nox2, and Nox4 with qPCR, ELISA and Western blotting.

An inducible model of low-grade IL-6 mediated inflammation employed AAV injection into abdominal fat, which has face validity for aging studies. The mouse IL-6 can be subcloned into an adeno-associated viral (AAV) vector, which has low immunogenicity and good safety profile, and transduces dividing and non-dividing cells, driving long-term gene expression (up to years) in tissues with low proliferation rates AAV8 or 9 can be used, as they mediate efficient and long term gene transfer to adipose tissues after administration to adult mice. Mice can be anesthetized with 3% isoflurane and receive 2×10¹⁰ to 1×10¹² viral genomes per mouse into intraepididymal fat. Each inguinal fat tissue receive four injections of 10 μL AAV solution using a Hamilton syringe. Serum levels of IL-6 are then be measured by ELISA to determine the time course of transgene expression and viral titrations.

d) Changes in the Activity of v-ATPase.

Similarly, lysosomal acidification by v-ATPase is also regulated by its assembly and trafficking. While it is not practical to measure lysosomal pH in vivo, possible changes in the assembly and targeting of v-ATPase that lead to lysosomal alkalinization can be examined by cell fractionation, western blotting, and immunofluorescence. When such changes are observed, the underlying mechanisms such as cysteine oxidation can be further examined

e) Changes in Lysosomal Activity in Tissue Lysates.

Oxidation modification of lysosome enzymes and subsequent inactivation can be detectable in tissue lysates. Hippocampus, cortex and other regions of brains can be dissected, frozen on dry ice and kept at −80° C. until homogenization with sonication in appropriate buffers. Cathepsin activities from homogenates of aged and inflammatory brains can be assayed in buffers with or without reducing reagents, using several fluorescence-based, commercially-available kits (Thermo Fisher) specific for CatD, CatB, and CatL activity. The results with cortical tissue of 19 month old vs 5 month old mice indicate that cathepsin activity can be reliably measured.

f) Western Blot Analyses of Autolytic Proteolytic Cleavage of CatD.

As CatD processing from its high molecular weight (HMW) pre-pro-forms, to its smaller active form requires lysosome acidification, Western blot analyses of tissue extracts can be carried out for various experimental groups, as shown in FIG. 7. Accumulation of un-cleaved HMW forms can be an additional index of lysosome dysfunction.

### 4. Example 4: IL-6 Deletion Improves Lysosomal Function

Multiple studies in both animal models and humans have shown that aging is associated with chronic, low-grade inflammation. In humans, increased circulating levels of the pro-inflammatory cytokine interleukin-6 (IL-6), specifically, is associated with age-related conditions and worse health outcomes in dozens of studies. There are numerous factors that can activate inflammatory pathways systemically, and in brain, in older adults, including chronic diseases such as diabetes, cardiovascular disease, hypertension, changes in hormone status, and even diet. More importantly, elevated IL-6 and Creactive protein, which is transcriptionally regulated by IL-6, predicts acute and chronic cognitive decline, mild cognitive impairment (MCI), Alzheimer's disease, and delirium in older adults. In support of the current proposal, clinical studies in even healthy older adults, have shown significant statistical correlations between elevated IL-6 and worse cognitive performance on neuropsychometric testing. In most of these studies, only IL-6, but not other pro-inflammatory mediators such as IL-1β and TNFα, was elevated.

To directly test the role of IL-6 in aging-impaired autophagy, brains from WT and IL-6_{-/-} mice can be used. It is shown herein that old IL-6_{-/-} mouse brain has lower Nox2 expression and activity in compared to WT. Previously cryopreserved brains from young (4 mo, n=8) and old (22 mo, n=12) WT, and young (n=10) and old (n=12) IL-6_{-/-} mice from this study can be examined Brains were PFA fixed, harvested and stored in cryoprotectant at −20°, so no new mice are proposed for these studies. Brain slices can be immunostained for p62, lamp2, parkin, pinkl, LC3B, lamp1, and cathepsins D and L. PV, p62 and MAP2 staining remained intact in these stored brains. If IL-6 is involved in lysosome inhibition, there can be fewer and/or smaller p62 aggregates than in WT brain (e.g. FIG. 5).

### 5. Example 5: Neuronal Deletion of Stat3 Improve Lysosomal Function?

These studies can be performed in two mouse lines generated specifically for this proposal, and in age- and gender matched WT mice, all on a C57BL6 background for more than 10 generations. The first line is a Thy1-YFP-Cre:tdTomato reporter mouse. These mice have constitutive expression of yellow fluorescent proteins (YFP) in 95% of hippocampal pyramidal and 90% of cortical neurons under control of the Thy1 promoter. One week of tamoxifen treatment in these mice, to activate the Cre-recombinase, produces high level expression of the red fluorescent protein tdTomato, in 95% of hippocampal neurons, and that there is nearly 100% tdTomato co-localization with YFP positive neurons. Therefore, mice can be studied one week after tamoxifen (Tam) treatment. These mice serve as a Tam-induction control for the final transgenic line, Thy1-YFP-Cre:Stat3flox/flox:tdTomato mice, which also have Tam-induced excision of Stat3 in neurons throughout hippocampus and cortex, again allowing for the ability to inducibly excise Stat3 in pyramidal and cortical neurons at any age.

Mice (equal male and female) can be studied at 5 months, 12 months, and 22 months of age, based on data obtained specifically for this grant. Equal numbers of males and females can be used. Nox2 is induced in aging brain in neurons and limited numbers of microglia during normal aging in WT mice, and further showed that IL-6 mediated this induction. Brain slices can be immunostained for Nox2 in WT and Tam-Thy1-YFPCre: Stat3floxed:tdTomato (TFtdTom) mice previously treated at 4-5 months of age with tamoxifen to excise Stat3 in hippocampal principal neurons. If Stat3 is necessary for induction of Nox expression, then a majority of neurons in old hippocampus and cortex express Nox(s) in WT animals, but not in the TFtdTom mice. In addition, Thy1-YFP-Cre:Stat3floxed mice without tdTom can be studied at the same time to confirm tdTom expression does not affect the results. To further test the role of Stat3 in Nox induction, IVC injection of IL-6 (1 μg in 4 μl saline) can be performed followed by immunostain for Nox2 48 hours later, based on studies, with the expectation that Nox(s) expression is not induced in neurons in Stat3 knockout mice. If inflammation still induces Nox2 expression in the absence of Stat3, this implicates transcriptional regulation of Nox through alternative factors.

(1) Data Analysis

The percent area of the hippocampus occupied by p62 aggregates, and the absolute number of aggregate clusters are compared between groups using ANOVA with Tukey's post-hoc, or if more than 4 groups are compared, a Bonferoni correction, with significance set at p<0.05. The size of aggregates, however, is a distribution, non-parametric analyses, such as Kruskal-Wallis or the Mann-Whitney rank sum tests, can be used to compare the distribution of sizes between groups. Both apocynin, by inhibiting Nox2 (and Nox1 if also involved), as well as the synthetic superoxide dismutase (SOD) mimetic (C3, which reduces levels of superoxide generated by Nox(s)) can both reduce the number and size of aggregates. The SOD mimetic can be more effective if Nox4 is also involved, as it can also reduce superoxide-to-hydrogen peroxide generation by Nox4. Even when activation of Nox(s) are not a dominant cause of lysosome failure, IL-6, acting through regulation of other inflammatory pathways, can be involved in impairing lysosome function in brain aging

### 6. Example 6: Inflammation Causes Lysosomal Dysfunction and Impaired Degradation of Lysosomal Contents

Genome wide association studies (GWAS) have repeatedly shown two networks of genes which confer increased risk for development of LO-NDs, and in some cases, earlier onset of disease. The first of these two networks includes genes associated with inflammation and regulation of innate immunity in the brain, with many genes specific to microglia, the resident monocytes of the brain. Risk genes identified in LO-Alzheimer's disease (LO-AD), specifically, include PU.1 (monocyte-lineage transcription factor), CD33, CR1, ABCA7, MS4A, and TREM2 (cell surface proteins on activated microglia), and the interleukin-6 receptor (IL-6R), among others. Additional GWAS in PD and FTD have identified many of the same risk genes. Gene array profiles from brains of normal, healthy older adults also showed increased expression of inflammatory genes, including CD33, IL-6R, and CR1 indicated persistent activation of innate immunity in brain for months after a systemic inflammatory exposure. Thus, multiple lines of evidence support a role for inflammatory activation innate immunity in the aging brain.

a) Inhibition of the v-ATPase Through Inflammatory Regulation of its Redox Site.

Exposure of mouse cortical cultures to IL-6 impairs lysosome degradation of proteins, including p62. Also, IL-6 can induce expression of inflammatory mediators, including TNFα, IL-6, IL-113, Nox2 and Nox4, through both canonical pTyr-Stat3 transcription, and through noncanonical Stat3 NFκB transcription. Here, primary mouse cultures can be prepared on coverslip dishes as described and treated with IL-6 (10 ng/ml and 100 ng/ml) or a vehicle controls (media), daily for 4 days. Bafilomycin (100 nM) can be included in some dishes as a positive control for direct v-ATPase inhibition. Cultures can be fixed with 4% paraformaldehyde (PFA), and immunostained. Based on the data, accumulation of large lamp1 and p62-positive lysosomes in IL-6 and BAF treated cells can be observed. To then assess the role of redox effects on the v-ATPase, cultures can be co-treated with one of three antioxidants, TEMPOL (general radical scavenging), the C3 compound (catalytic superoxide dismutase, to reduce Nox-derived superoxide, regardless of isoform(s) which are contributing), and N-acetyl cysteine (to increase glutathione levels). When the v-ATPase is inhibited through its redox site, antioxidant treatment can prevent accumulation of undigested contents (p62, lamp1), and prevent enlargement of lysosomes. In addition, if lysosome acidification and therefore cathepsin activity is already impaired in cultures treated with IL-6 via redox inactivation of v-ATPase, then BAF can not further worsen lysosome function because the v-ATPase is already inhibited. Conversely, treatment with antioxidants can preserve v-ATPase activity, allowing BAF to produce inhibitory effects on the v-ATPase and lysosome cargo degradation.

If the above experiments implicate v-ATPase as targets of Nox-derived reactive oxygen species (ROS) in inducing lysosomal dysfunction in neuronal cultures, the mechanisms leading to inactivation of v-ATPase can be further examined V-ATPase is a multisubunit complex composed of a peripheral V1 domain that hydrolyzes ATP and an integral V0 domain that translocates protons. In mammalian cells, PI-3 kinase, ERK, and mTOR promote its assembly while PKA activation promotes its translocation to plasma membrane. It is also extremely sensitive to oxidative inhibition, and in mammals, this appears to be through oxidation of Cys254 or Cys277 of the ATP6VA1 subunit, although the exact mechanism is still debatable. High level of ROS also lead to the disassembly of the peripheral V1 domain from vesicles in non-mammalian cells. To determine v-ATPase changes in cultures induced by Nox activation and proposed treatments above, lysosomes can be isolated using a kit (Sigma Aldrich). ATPase activity can be measured by phosphate release quantified by colorimetric reaction with malachite green (Sigma Aldrich). v-ATPase activity can be defined by the difference in the absence and presence of bafilomycin. If inactivation is detected and is reversed by pretreatment with DTT, it is likely that inactivation is due to cysteine oxidation or formation of disulfide bonds.

b) Direct Chemical Alkalinization of Lysosomes Results in Inhibition of Cathepsin Acid Protease Activity.

Similarly, cultures can be exposed to IL-6 as above and the effect on lysosome pH can be monitored. Primary neuronal cultures can be grown on glass bottom 96 well plates, treated with IL-6 to induce Nox activation, then loaded with LysoTracker green (ThermoFisher, L7526), whose accumulation in lysosomes depends on lysosomal pH. Fluorescence intensity can be quantified by fluorescence plate reader, or alternatively imaging with confocal microscopy. Activation of Nox(s) can lead to alkalinization of lysosomes, and produce a decrease in fluorescence which can be reversed by Nox inhibition and ROS scavengers. Accumulation of LysoTracker dyes can also be affected by conditions such as cytosolic pH, and to rule out such possibilities, ratiometric probes such as LysoSensor™ Yellow/Blue DND-160 can be also used to repeat the above experiments whenever feasible. Neurons can uptake dextran-conjugated Oregon green and rhodamine into lysosomes after extended incubation, and the ratio of Oregon green over rhodamine can also reflect changes in lysosomal pH. The above experiments can be repeated using these dyes as they label lysosomes via different pathways. For some dishes, after imaging cells can be fixed, immunostained for lysosome markers, including lamp1, and the fields relocated to further confirm the identity of lysosomes which had previously been imaged for pH changes.

To evaluate lysosomal protein degradation in cultured cells, which is dependent on both lysosomal pH and protease activity, cells can be incubated with DQ Green BSA (ThermoFisher Scientific) then washed, and fluorescence can be monitored at different time points. Increased fluorescence correlates with the degradation of DQ Green BSA. As this assay is dependent on endocytosis, effects of treatments on endocytosis can also be simultaneously assessed with dextran conjugated Rhodamine. Alternatively, cell permeable substrates for different lysosome proteases such as Rhodamine 110-based bis-peptide substrates (ThermoFisher Scientific, for example, Bis-(CBZ-Arg)-R110 for serine protease, Bis-(CBZ-Phe-Arg)-R110 for cathepsin B and L) can be used to monitor their degradation in cells. It is anticipated that these assays are dependent on both lysosomal pH and protease activities; the latter can be further assessed with activity dependent probes such as BMV109 (Vergent Bioscience), a pan-cathepsin probe that has been used to detect cathepsin activity in cell lysates, intact cells, and in vivo3. The fluorescence can be detected by either microscopy or fluorescence plate reader. Together, these assays, combined with experiments described herein provide insights into whether the changes in protease degradation are due to altered pH or protease activities.

(1) Analysis and Interpretation:

When Nox is responsible for alkalinization of lysosomes, higher intra-lysosomal pH after Nox induction is observed, which can be prevented or normalized by Nox inhibition. ANOVA with Bonferonni post-hoc for multiple comparisons can be used to compare each Nox induction treatment with controls, and with Nox inhibition. Lack of change in lysosomal pH with Nox induction implicates either an inadequate level or duration of induction, or an alternative mechanism for lysosome failure, as can be explored below. The studies herein show higher pH with Nox induction, so based on these data, Nox-dependent pH changes can be confirmed. Likewise, if pH changes do not predict effects on protease efficiency, then alternative mechanisms are likely and are explored below.

c) Oxidative Modification of Intra-Lysosomal Proteins, Including Cathepsins, Causes Impaired Proteolysis.

Cathepsins, particularly cysteine proteases such as cathepsin B, L and S, have cysteine residues that are susceptible to oxidation, resulting in their inactivation. To assess the effects of ROS on the activities of cathepsins, cultures exposed can be harvested in buffers with or without reducing reagents, and have cathepsin activity measured using appropriate substrates [Z-FR-AMC from Anespec for cathepsin B (sensitive portion to CA-074) and L (insensitive to CA-074), cathespin S substrate from EMD Millipore, cathepsin D&E substrate from Enzo]. Aspartyl cathespins (CatD or CatE) can be less impacted by possible oxidation, while activities of cysteinyl cathepsins B, L and S can be preferentially impaired.

(1) Human Neuronal Cultures from iPSC Cells.

Neuronal cultures derived from the well-characterized human inducible pleuripotent stem cell (iPSC) line, CC-3, were established and can be used confirm key findings from the mouse culture experiments. Human neurons differentiated from the CC-3 line are 60% excitatory, and 40% inhibitory (5% PV-positive). Because little is known about the concentrations of drugs needed to modulate inflammation in cultured human neurons, dose-finding experiments can be carried out first. Differentiation of iPSCs to neurons takes 5 weeks, so these cultures are best used to confirm key observations from mouse cultures. One additional compelling rationale for use of human neurons is that the Nox5 gene is present in humans, but not rodents. Evidence indicates that Nox5, which is Ca2+-dependent can be an important source of ROS in humans, and Nox5 is known to be expressed in human neurons. To determine whether Nox5 can affect lysosomes, the cell-permeable Ca2+ chelator, BAPTAAM (Thermofisher) can be used to inhibit Nox5. These studies provide an exciting new approach to how lysosome function is impacted by inflammation in human neurons.

### 7. Example 7: Role of Nox Activation, IL-6, and Stat3 Activation in Lysosome Dysfunction in Neuronal Cultures

To implicate Nox in lysosome impairment, cells treated with IL-6 (10 and 100 ng/ml) can be co-treated with 1) the pro-drug Nox inhibitor, apocynin (100 μM), 2) a blocking antibody to p22phox, which selectively inhibits Nox, or 3) p22phox shRNA as described. In human neurons, the effects of Nox5 shRNA can be tested. Nox(s) induction can be monitored by immunostaining for Nox(s), and by Western blots for Nox(s) subunit expression with antibodies that were validated for mouse and/or human samples. Nox activity is monitored by EPR detection of superoxide generation. To determine the role of IL-6, blocking antibodies to IL-6 can be administered with drugs, as described.

To determine the role of Stat3 in lysosome dysfunction, neurons can be prepared from mice with Tamoxifen (TAM)-inducible deletion of Stat3 (“TF” mice). Stat3 is deleted from 95% of hippocampal pyramidal neurons. These mice are maintained in the PI's colony. Hippocampal cultures from these mice and WT C57BL6 mice can be prepared. WT and TF cultures are treated with either TAM or corn oil vehicle 3 days after plating to excise Stat3 in TF but not WT cultures. Cultures are then studied at 14 days in culture, using exposure to NMDA (10 μM) for 22 hours to induce Nox activation as described. If Stat3 is necessary for lysosome inhibition, then TF cultures treated with TAM, but not WT cultures, or cultures treated with vehicle, has improved lysosome function and less Nox activation. Based on that, Stat3 deletion can still reduce inflammatory signaling and Nox induction. Of note, Jak2 inhibitors prevent only tyrosine phosphorylation, so the actions of U-Stat3 or p-SerStat3 is not blocked. AG490 is a Jak inhibitor which prevents Stat3 activation, and this can be used a second means to inhibit Stat3, although it does not inhibit all Stat3 activities, as Stat3 can be phosphorylated on serine as well as tyrosine resides, so the genetic deletion experiments can provide different results than the AG0490 treatment.

a) Data Analysis and Interpretation.

If oxidation inactivates cathepsins by the formation of disulfide bonds or sulfenylation, their activities can be restored by the inclusion of reducing reagents. Treating cells with antioxidants can at least partially reverse inflammation-induced inactivation of cathepsins. ANOVA with the Bonferoni post-hoc can determine whether direct redox effects are inhibiting cathepsin activity.

### 8. Example 8: Undegraded Lysosomal Cargo, Including Active Proteases, Injures Nearby Neurons, and Whether Undigested Lysosomal Material is Pro-Inflammatory

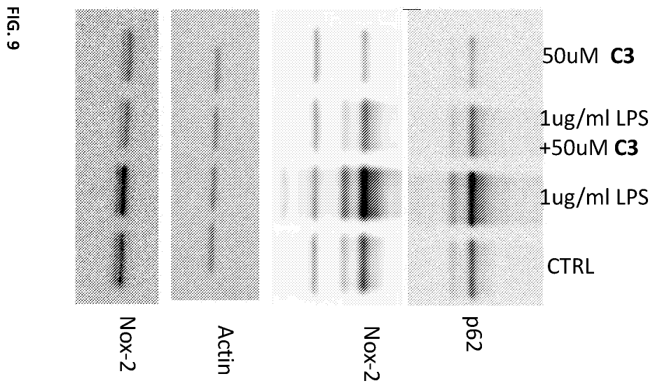

It is clear from studies outside the nervous system, such as in heart and bone, that ineffective lysosomes can release active proteases into the extracellular space, which then destroys nearby tissue. The data also indicate that there is injury to neuronal fibers in proximity to aggregates of lysosomal material (FIG. 8). Mice expressing the tdTom red fluorescent reporter protein in subsets of neurons can be used to visualize neuronal processes and synapses by confocal fluorescence microscopy. Regression analyses can be performed for young and old mice, and for mice treated with apocynin, to determine whether proximity to aggregates predicts fiber loss, using a test for non-parametric distributions, available in both Sigmastat and the Excel statistics package. The relationship between local aggregates and fiber loss can be graphed and linear regression analysis performed to determine a correlation coefficient (r), which can be compared statistically across groups. If extruded lysosomal material causes damage to local neuronal fibers, then r approaches 1.0, but if not, then r approaches 0.5. Similarly, it is shown herein whether proximity to aggregates increases local inflammation, assessed as microglial and astrocyte activation.

a) Effects of Lysosome Dysfunction on Integrity of Neurons.

To allow easy visualization of single neuronal axons, dendrites and synapses—all potential targets of injury—a PV-Cre:tdTomato reporter mice can be used, which express the red fluorescent protein tdTomato (tdTom) in Palvalbumin-expressing inhibitory (PV) neurons. The choice of these mice was made because these neurons are sufficiently rare that it is feasible to see individual neurons, their cell bodies, and their processes, in detail. Published protocols can be used to analyze neuronal fiber density and synapse numbers, using immunostaining for MAP-2, β-Tub, SMI32 (fibers), and PSD95, synaptophysin (synapses).

When impaired lysosome function, and extruded still active cathepsins are responsible for bystander injury to neuronal fibers/synapses, then treatments which reduce aggregates can reduce damage only for neurons near the aggregates. Where treatments provide a general reduction in fiber loss, that argues against aggregates as responsible for fiber damage.

b) Lysosomal Aggregates Induce a Local Inflammatory Reaction

Activation of astrocytes and microglia can be analyzed as two measures of inflammation. To evaluate inflammatory activation of astrocytes and microglia near extruded lysosomes, IF can be carried out for GFAP and for Iba1, and look for increased GFAP-positive astrocytes near aggregates indicating a local activation of astrocytes. Likewise, slices can be immunostained for Iba1 to look for a local microglial inflammatory response. Local induction of inflammation can be monitored similarly by looking at microglial and astrocyte activation, and induction of pro-inflammatory markers including HLA-DR4α, MHC II, and Iba-1. Astrocyte activation can be followed by GFAP expression. The GFAP-tdTom and the Cx3cr1-eGFP reporter mice can be used and brain slices from young and old mice imaged if needed. Increased activation of both microglia and astrocytes is known to occur in the aging brain, including in humans. However, to date, it is unknown to what extent extruded lysosomal contents and undigested lysosomal contents activate local inflammation in brain aging. Also determined herein is the statistical correlation between astrocyte activation, defined as GFAP and IL-6 expression, and the presence of a p62-positive aggregate within 20 μm (roughly 2 cell diameters). Likewise, a significant correlation between p62 aggregate clusters and induction of pro-inflammatory markers on microglia, including IL-6 receptors, MHC II, and Iba-1, indicates a local inflammatory response to aggregates.

### 9. Example 9: Rescue of Lysosomal Function

To show the efficacy of NOX inhibition to rescue lysosome function, J774A.1 cells were treated with the pro-inflammatory molecule LPS to impair lysosome function. As shown in FIG. 9, upon stimulation with 1 μg/mL LPS p62 and NOX 2 were upregulated relative controls indicating lysosome impairment. However, cells treated with 1 μg/mL LPS in the presence of 50 μM C3 showed no increase in p62 or NOX2 i.e., rescuing lysosome function.

### C. References

Abd El Mohsen, M. M., Iravani, M. M., Spencer, J. P., Rose, S., Fahim, A. T., Motawi, T. M., Ismail, N. A., and Jenner, P. (2005). Age-associated changes in protein oxidation and proteasome activities in rat brain: modulation by antioxidants. Biochem Biophys Res Commun 336, 386-391.
Abeliovich, A. & Gitler, A. D. Defects in trafficking bridge Parkinson's disease pathology and genetics. Nature 539, 207-216, doi:10.1038/nature20414 (2016).
Abeliovich, A. & Rhinn, H. Parkinson's disease: Guilt by genetic association. Nature 533, 40-41, doi:10.1038/nature17891 (2016).
Abeliovich, A. Parkinson's disease: pro-survival effects of PINK1. Nature 448, 759-760, doi:10.1038/448759a (2007).
Akiyama, H., Kameyama, M., Akiguchi, I., Sugiyama, H., Kawamata, T., Fukuyama, H., Kimura, H., Matsushita, M., and Takeda, T. (1986). Periodic acid-Schiff (PAS)-positive, granular structures increase in the brain of senescence accelerated mouse (SAM). Acta Neuropathol 72, 124-129.
Ali, S. S. et al. A biologically effective fullerene (C60) derivative with superoxide dismutase mimetic properties. Free Radic Biol Med 37, 1191-1202, (2004).
Ali, S. S. et al. Initial evidence linking synaptic superoxide production with poor short-term memory in aged mice. Brain Res 1368, 65-70, doi:10.1016/j.brainres.2010.11.009 (2011).
Ali, S. S., Hardt, J. I. & Dugan, L. L. SOD activity of carboxyfullerenes predicts their neuroprotective efficacy: a structure-activity study. Nanomedicine: nanotechnology, biology, and medicine 4, 283-294, doi:10.1016/j.nano.2008.05.003 (2008).
Bagh, M. B. et al. Misrouting of v-ATPase subunit V0a1 dysregulates lysosomal acidification in a neurodegenerative lysosomal storage disease model. Nature communications 8, 14612, doi:10.1038/ncomms14612 (2017).
Bao, J. X., Jin, S., Zhang, F., Wang, Z. C., Li, N., and Li, P. L. (2010). Activation of membrane NADPH oxidase associated with lysosome-targeted acid sphingomyelinase in coronary endothelial cells. Antioxid Redox Signal 12, 703-712.
Barth, S., Glick, D. & Macleod, K. F. Autophagy: assays and artifacts. J Pathol 221, 117-124, doi:10.1002/path.2694 (2010).
Bartlett, B. J. et al. p62, Ref(2)P and ubiquitinated proteins are conserved markers of neuronal aging, aggregate formation and progressive autophagic defects. Autophagy 7, 572-583 (2011).
Bedard, K. & Krause, K. H. The NOX family of ROS-generating NADPH oxidases: physiology and pathophysiology. Physiol Rev 87, 245-313 (2007).
Bedard, K., Lardy, B. & Krause, K.-H. NOX family NADPH oxidases: Not just in mammals. Biochimie 89, 1107-1112, (2007).
Behrens, M. M. et al. Ketamine-induced loss of phenotype of fast-spiking interneurons is mediated by NADPH-oxidase. Science 318, 1645-1647, doi:10.1126/science.1148045 (2007).
Behrens, M. M., Ali, S. S., and Dugan, L. L. (2008). Interleukin-6 mediates the increase in NADPHoxidase in the ketamine model of schizophrenia. J Neurosci 28, 13957-13966.
Bento, C. F. et al. Mammalian Autophagy: How Does It Work? Annu Rev Biochem 85, 685-713, doi:10.1146/annurev-biochem-060815-014556 (2016).
Bento, C. F., Ashkenazi, A., Jimenez-Sanchez, M. & Rubinsztein, D. C. The Parkinson's disease-associated genes ATP13A2 and SYT11 regulate autophagy via a common pathway. Nature communications 7, 11803, doi:10.1038/ncomms11803 (2016).
Bettcher, B. M. et al. Interleukin-6, age, and corpus callosum integrity. PLoS One 9, e106521, doi:10.1371/journal.pone.0106521 (2014).
Bordi, M. et al. Autophagy flux in CA1 neurons of Alzheimer hippocampus: Increased induction overburdens failing lysosomes to propel neuritic dystrophy. Autophagy 12, 2467-2483, doi:10.1080/15548627.2016.1239003 (2016).
Canzoniero, L. M. et al. nNOS(+) striatal neurons, a subpopulation spared in Huntington's Disease, possess functional NMDA receptors but fail to generate mitochondrial ROS in response to an excitotoxic challenge. Frontiers in physiology 4, 112, doi:10.3389/fphys.2013.00112 (2013).
Chapin, H. C., Okada, M., Merz, A. J., and Miller, D. L. (2015). Tissue-specific autophagy responses to aging and stress in C. elegans. Aging 7, 419-434.
Cheng, G., Cao, Z., Xu, X., Meir, E. G. V. & Lambeth, J. D. Homologs of gp9lphox: cloning and tissue expression of Nox3, Nox4, and Nox5. Gene 269, 131-140 (2001).
Chung, W. S., Clarke, L. E., Wang, G. X., Stafford, B. K., Sher, A., Chakraborty, C., Joung, J., Foo, L. C., Thompson, A., Chen, C., et al. (2013). Astrocytes mediate synapse elimination through MEGF10 and MERTK pathways. Nature 504, 394-400.
Conti, F. et al. Biological therapies in rheumatic diseases. Clin Ter 164, e413-428, doi:10.7417/CT.2013.1622 (2013).
Corrada, M. M., Brookmeyer, R., Paganini-Hill, A., Berlau, D. & Kawas, C. H. Dementia incidence continues to increase with age in the oldest old: the 90+ study. Ann Neurol 67, 114-121, doi:10.1002/ana.21915 (2010).
Cotter, K., Stransky, L., McGuire, C. & Forgac, M. Recent Insights into the Structure, Regulation, and Function of the V-ATPases. Trends Biochem Sci 40, 611-622, doi:10.1016/j.tibs.2015.08.005 (2015).
Cuervo, A. M. & Macian, F. Autophagy and the immune function in aging. Curr Opin Immunol 29, 97-104, doi:10.1016/j.coi.2014.05.006 (2014).
Cuervo, A. M. Autophagy and aging: keeping that old broom working. Trends Genet 24, 604-612, doi:10.1016/j.tig.2008.10.002 (2008).
Dahal, D. et al. An NIR-emitting lysosome-targeting probe with large Stokes shift via coupling cyanine and excited-state intramolecular proton transfer. Chem Commun (Camb) 53, 3697-3700, doi:10.1039/c7cc00700k (2017).
Dik, M. G. et al. Serum inflammatory proteins and cognitive decline in older persons. Neurology 64, 1371-1377 (2005).
Doehner, J., Genoud, C., Imhof, C., Krstic, D., and Knuesel, I. (2012). Extrusion of misfolded and aggregated proteins—a protective strategy of aging neurons? Eur J Neurosci 35, 1938-1950.
Doehner, J., Madhusudan, A., Konietzko, U., Fritschy, J. M., and Knuesel, I. (2010). Co-localization of Reelin and proteolytic AbetaPP fragments in hippocampal plaques in aged wild-type mice. J Alzheimers Dis 19, 1339-1357.
Dschida, W. J. & Bowman, B. J. The vacuolar ATPase: sulfite stabilization and the mechanism of nitrate inactivation. J Biol Chem 270, 1557-1563 (1995).
Dugan, L. L. et al. IL-6 mediated degeneration of forebrain GABAergic interneurons and cognitive impairment in aged mice through activation of neuronal NADPH oxidase. PLoS ONE 4, e5518 (2009).
Dugan, L. L. et al. Mitochondrial production of reactive oxygen species in cortical neurons following exposure to N-methyl-D-aspartate. J Neurosci 15, 6377-6388 (1995).
Edgington-Mitchell, L. E., Bogyo, M. & Verdoes, M. Live Cell Imaging and Profiling of Cysteine Cathepsin Activity Using a Quenched Activity-Based Probe. Methods Mol Biol 1491, 145-159, doi:10.1007/978-1-4939-6439-O_11 (2017).
El Chemaly, A., Nunes, P., Jimaj a, W., Castelbou, C., and Demaurex, N. (2014). Hvl proton channels differentially regulate the pH of neutrophil and macrophage phagosomes by sustaining the production of phagosomal ROS that inhibit the delivery of vacuolar ATPases. J Leukoc Biol 95, 827-839.
Feng, Y., and Forgac, M. (1994). Inhibition of vacuolar H(+)-ATPase by disulfide bond formation between cysteine 254 and cysteine 532 in subunit A. J Biol Chem 269, 13224-13230.
Fiandaca, M. S., Kapogiannis, D., Mapstone, M., Boxer, A., Eitan, E., Schwartz, J. B., Abner, E. L., Petersen, R. C., Federoff, H. J., Miller, B. L., et al. (2015). Identification of preclinical Alzheimer's disease by a profile of pathogenic proteins in neurally derived blood exosomes: A case-control study. Alzheimers Dement 11, 600-607 e601.
Flaig, T. et al. Tocilizumab-induced pancreatitis: case report and review of data from the FDA Adverse Event Reporting System. J Clin Pharm Ther 41, 718-721, doi:10.1111/jcpt.12456 (2016).
Franceschi, C. & Campisi, J. Chronic inflammation (inflammaging) and its potential contribution to age-associated diseases. J Gerontol A Biol Sci Med Sci 69 Suppl 1, S4-9, doi:10.1093/gerona/g1u057 (2014).
Freund, A., Orjalo, A. V., Desprez, P. Y. & Campisi, J. Inflammatory networks during cellular senescence: causes and consequences. Trends Mol Med 16, 238-246, doi:10.1016/j.molmed.2010.03.003 (2010).
Gan-Or, Z., Dion, P. A. & Rouleau, G. A. Genetic perspective on the role of the autophagy-lysosome pathway in Parkinson disease. Autophagy 11, 1443-1457, doi:10.1080/15548627.2015.1067364 (2015).
Gan-Or, Z., Dion, P. A., and Rouleau, G. A. (2015). Genetic perspective on the role of the autophagylysosome pathway in Parkinson disease. Autophagy 11, 1443-1457.
Gao, J. et al. Glucocorticoid impairs cell-cell communication by autophagy-mediated degradation of connexin 43 in osteocytes. Oncotarget 7, 26966-26978, doi:10.18632/oncotarget.9034 (2016).
Garcia-Prat, L., Martinez-Vicente, M., Perdiguero, E., Ortet, L., Rodriguez-Ubreva, J., Rebollo, E., Ruiz-Bonilla, V., Gutarra, S., Ballestar, E., Serrano, A. L., et al. (2016). Autophagy maintains stemness by preventing senescence. Nature 529, 37-42.
Gavilán, E., Pintado, C., Gavilan, M. P., Daza, P., Sanchez-Aguayo, I., Castaflo, A., and Ruano, D. (2015). Age-related dysfunctions of the autophagy lysosomal pathway in hippocampal pyramidal neurons under proteasome stress. Neurobiology of Aging 36, 1953-1963.
Giuliani, N. et al. Serum interleukin-6, soluble interleukin-6 receptor and soluble gp130 exhibit different patterns of age- and menopause-related changes. Exp Gerontol 36, 547-557 (2001).
Godbout, J. P. & Johnson, R. W. Age and neuroinflammation: a lifetime of psychoneuroimmune consequences. Neurol Clin 24, 521-538 (2006).
Godbout, J. P. & Johnson, R. W. Interleukin-6 in the aging brain. J Neuroimmunol 147, 141-144 (2004).
Goetzl, E. J., Boxer, A., Schwartz, J. B., Abner, E. L., Petersen, R. C., Miller, B. L., and Kapogiannis, D. (2015). Altered lysosomal proteins in neural-derived plasma exosomes in preclinical Alzheimer disease. Neurology 85, 40-47.
Goldberg, M. P., Strasser, U. & Dugan, L. L. Techniques for assessing neuroprotective drugs in vitro. Int Rev Neurobiol 40, 69-93 (1997).
Gottlieb, R. A., Andres, A. M., Sin, J. & Taylor, D. P. Untangling autophagy measurements: all fluxed up. Circ Res 116, 504-514, doi:10.1161/CIRCRESAHA.116.303787 (2015).
Griciuc, A. et al. Alzheimer's disease risk gene CD33 inhibits microglial uptake of amyloid beta. Neuron 78, 631-643, doi:10.1016/j.neuron.2013.04.014 (2013).
Guenette, S. Y. (2003). Astrocytes: a cellular player in Abeta clearance and degradation. Trends Mol Med 9, 279-280.
Gurney, M. A. et al. Measuring cardiac autophagic flux in vitro and in vivo. Methods in molecular biology 1219, 187-197, doi:10.1007/978-1-4939-1661-0_14 (2015).
Hall, D., Han, S. & Dugan, L. L. In vivo optical imaging of dihydroethidium oxidation in the mouse brain employing fluorescence intensity and lifetime contrast. (2009).
Harold, D. et al. Genome-wide association study identifies variants at CLU and PICALM associated with Alzheimer's disease. Nat Genet 41, 1088-1093, doi:10.1038/ng.440 (2009).
Heid, M. E., Keyel, P. A., Kamga, C., Shiva, S., Watkins, S. C., and Salter, R. D. (2013). Mitochondrial reactive oxygen species induces NLRP3-dependent lysosomal damage and inflammasome activation. J Immunol 191, 5230-5238.
Hohn, A., Konig, J., and Grune, T. (2013). Protein oxidation in aging and the removal of oxidized proteins. J Proteomics 92, 132-159.
Hohn, A., Konig, J., and Jung, T. (2016). Metabolic Syndrome, Redox State, and the Proteasomal System. Antioxid Redox Signal 25, 902-917.
Howcroft, T. K. et al. The role of inflammation in age-related disease. Aging (Albany N. Y.) 5, 84-93, doi:10.18632/aging.100531 (2013).
Huang, K. L. e. a. A common haplotype lowers PU.1 expression in myeloid cells and delays onset of Alzheimer's disease. Nature Neuroscience In Press., (2017).
Iking-Konert, C. et al. ROUTINE-a prospective, multicentre, non-interventional, observational study to evaluate the safety and effectiveness of intravenous tocilizumab for the treatment of active rheumatoid arthritis in daily practice in Germany Rheumatology 55, 624-635, doi:10.1093/rheumatology/kev372 (2016).
Jaishy, B., Zhang, Q., Chung, H. S., Riehle, C., Soto, J., Jenkins, S., Abel, P., Cowart, L. A., Van Eyk, J. E., and Abel, E. D. (2015). Lipid-induced NOX2 activation inhibits autophagic flux by impairing lysosomal enzyme activity. J Lipid Res 56, 546-561.
Jones, L. et al. Genetic evidence implicates the immune system and cholesterol metabolism in the aetiology of Alzheimer's disease. PLoS One 5, e13950, doi:10.1371/journal.pone.0013950 (2010).
Jordans, S., Jenko-Kokalj, S., Kuhl, N. M., Tedelind, S., Sendt, W., Bromme, D., Turk, D., and Brix, K. (2009). Monitoring compartment-specific substrate cleavage by cathepsins B, K, L, and S at physiological pH and redox conditions. BMC Biochem 10, 23.
Jucker, M., Walker, L. C., Martin, L. J., Kitt, C. A., Kleinman, H. K., Ingram, D. K., and Price, D. L. (1992). Age-associated inclusions in normal and transgenic mouse brain. Science 255, 1443-1445.
Jucker, M., Walker, L. C., Schwarb, P., Hengemihle, J., Kuo, H., Snow, A. D., Bamert, F., and Ingram, D. K. (1994). Age-related deposition of glia-associated fibrillar material in brains of C57BL/6 mice. Neuroscience 60, 875-889.
Jun, G., Naj, A. C., Beecham, G. W., Wang, L. S., Buros, J., Gallins, P. J., Buxbaum, J. D., Ertekin-Taner, N., Fallin, M. D., Friedland, R., et al. (2010). Meta-analysis confirms CR1, CLU, and PICALM as alzheimer disease risk loci and reveals interactions with APOE genotypes. Arch Neurol 67, 1473-1484.
Kawahara, T., Ritsick, D., Cheng, G. & Lambeth, J. D. Point mutations in the proline-rich region of p22phox are dominant inhibitors of Nox1- and Nox2-dependent reactive oxygen generation. J Biol Chem 280, 31859-31869 (2005).
Klionsky, D. J. et al. Guidelines for the use and interpretation of assays for monitoring autophagy. Autophagy 8, 445-544 (2012).
Klionsky, D. J. et al. Guidelines for the use and interpretation of assays for monitoring autophagy (3rd edition). Autophagy 12, 1-222, doi:10.1080/15548627.2015.1100356 (2016).
Knuesel, I., Nyffeler, M., Mormede, C., Muhia, M., Meyer, U., Pietropaolo, S., Yee, B. K., Pryce, C. R., LaFerla, F. M., Marighetto, A., et al. (2009). Age-related accumulation of Reelin in amyloid-like deposits. Neurobiol Aging 30, 697-716.
Kop, W. J. et al. Inflammation and coagulation factors in persons >65 years of age with symptoms of depression but without evidence of myocardial ischemia. Am J Cardiol 89, 419-424 (2002).
Krabbe, K. S., Pedersen, M. & Bruunsgaard, H. Inflammatory mediators in the elderly. Exp Gerontol 39, 687-699 (2004).
Kukull, W. A. et al. Dementia and Alzheimer disease incidence: a prospective cohort study. Arch Neurol 59, 1737-1746 (2002).
Kuo, H., Ingram, D. K., Walker, L. C., Tian, M., Hengemihle, J. M., and Jucker, M. (1996). Similarities in the age-related hippocampal deposition of periodic acid-schiff-positive granules in the senescence accelerated mouse P8 and C57BL/6 mouse strains. Neuroscience 74, 733-740.
Lambert, J. C. et al. Implication of the immune system in Alzheimer's disease: evidence from genome-wide pathway analysis. Journal of Alzheimer's disease: JAD 20, 1107-1118, doi:10.3233/JAD-2010-100018 (2010).
Lambert, J. C. et al. Meta-analysis of 74,046 individuals identifies 11 new susceptibility loci for Alzheimer's disease. Nat Genet 45, 1452-1458, doi:10.1038/ng.2802 (2013).
Lambeth, J. D. NOX enzymes and the biology of reactive oxygen. Nat Rev Immunol 4, 181-189 (2004).
Lane, N. E. & Yao, W. Glucocorticoid-induced bone fragility. Ann N Y Acad Sci 1192, 81-83, doi:10.1111/j.1749-6632.2009.05228.x (2010).
Lane, N. E. et al. Glucocorticoid-treated mice have localized changes in trabecular bone material properties and osteocyte lacunar size that are not observed in placebo-treated or estrogen-deficient mice. Journal of bone and mineral research: the official journal of the American Society for Bone and Mineral Research 21, 466-476, doi:10.1359/JBMR.051103 (2006).
LeBrasseur, N. K., Tchkonia, T. & Kirkland, J. L. Cellular Senescence and the Biology of Aging, Disease, and Frailty. Nestle Nutr Inst Workshop Ser 83, 11-18, doi:10.1159/000382054 (2015).
Lefaki, M., Papaevgeniou, N., and Chondrogianni, N. (2017). Redox regulation of proteasome function. Redox Biol 13, 452-458.
Li, X., Gulbins, E., and Zhang, Y. (2012). Oxidative stress triggers Ca-dependent lysosome trafficking and activation of acid sphingomyelinase. Cell Physiol Biochem 30, 815-826.
Licastro, F., Grimaldi, L. M., Bonafe, M., Martina, C., Olivieri, F., Cavallone, L., Giovanietti, S., Masliah, E., and Franceschi, C. (2003). Interleukin-6 gene alleles affect the risk of Alzheimer's disease and levels of the cytokine in blood and brain. Neurobiol Aging 24, 921-926.
Lindmo, K. et al. The PI 3-kinase regulator Vps15 is required for autophagic clearance of protein aggregates. Autophagy 4, 500-506 (2008).
Lipinski, M. M., Zheng, B., Lu, T., Yan, Z., Py, B. F., Ng, A., Xavier, R. J., Li, C., Yankner, B. A., Scherzer, C. R., et al. (2010). Genome-wide analysis reveals mechanisms modulating autophagy in normal brain aging and in Alzheimer's disease. Proc Natl Acad Sci USA 107, 14164-14169.
Loos, B., du Toit, A. & Hofmeyr, J. H. Defining and measuring autophagosome flux-concept and reality. Autophagy 10, 2087-2096, doi:10.4161/15548627.2014.973338 (2014).
Lu, T. et al. Gene regulation and DNA damage in the ageing human brain. Nature 429, 883-891 (2004).
Lubbe, S., and Morris, H. R. (2014). Recent advances in Parkinson's disease genetics. J Neurol 261, 259-266.
Maggio, M., Guralnik, J. M., Longo, D. L., and Ferrucci, L. (2006). Interleukin-6 in aging and chronic disease: a magnificent pathway. J Gerontol A Biol Sci Med Sci 61, 575-584.
Manich, G., del Valle, J., Cabezon, I., Camins, A., Pallas, M., Pelegri, C., and Vilaplana, J. (2014). Presence of a neo-epitope and absence of amyloid beta and tau protein in degenerative hippocampal granules of aged mice. Age (Dordr) 36, 151-165.
Mann, D. M., Yates, P. O., and Stamp, J. E. (1978). The relationship between lipofuscin pigment and ageing in the human nervous system. J Neurol Sci 37, 83-93.
Martin, I. & Grotewiel, M. S. Oxidative damage and age-related functional declines. Mech Ageing Dev 127, 411-423 (2006).
McAuley, T., Yap, M., Christ, S. E. & White, D. A. Revisiting inhibitory control across the life span: insights from the ex-Gaussian distribution. Dev Neuropsychol 29, 447-458 (2006).
McDonald, J. H. & Dunn, K. W. Statistical tests for measures of colocalization in biological microscopy. J Microsc 252, 295-302, doi:10.1111/jmi.12093 (2013).
McLaughlin, M. & Ostor, A. Safety of subcutaneous versus intravenous tocilizumab in combination with traditional disease-modifying antirheumatic drugs in patients with rheumatoid arthritis. Expert opinion on drug safety 14, 429-437, doi:10.1517/14740338.2015.998198 (2015).
Medina, D. L., Fraldi, A., Bouche, V., Annunziata, F., Mansueto, G., Spampanato, C., Puri, C., Pignata, A., Martina, J. A., Sardiello, M., et al. Transcriptional activation of lysosomal exocytosis promotes cellular clearance. Dev Cell 21, 421-430.
Mehra, S. et al. Clinical significance of cathepsin L and cathepsin B in dilated cardiomyopathy. Mol Cell Biochem 428, 139-147, doi:10.1007/s11010-016-2924-6 (2017).
Mehrjoo, Z. et al. Association Study of the TREM2 Gene and Identification of a Novel Variant in Exon 2 in Iranian Patients with Late-Onset Alzheimer's Disease. Med Princ Pract 24, 351-354, doi:10.1159/000430842 (2015).
Menzies, F. M. et al. Autophagy and Neurodegeneration: Pathogenic Mechanisms and Therapeutic Opportunities. Neuron 93, 1015-1034, doi:10.1016/j.neuron.2017.01.022 (2017).
Mooijaart, S. P., Sattar, N., Trompet, S., Lucke, J., Stott, D. J., Ford, I., Jukema, J. W., Westendorp, R. G., de Craen, A. J., and Group, P. S. (2013). Circulating interleukin-6 concentration and cognitive decline in old age: the PROSPER study. J Intern Med 274, 77-85.
Nagele, R. G., D'Andrea, M. R., Lee, H., Venkataraman, V., and Wang, H.-Y. (2003). Astrocytes
Naj, A. C. et al. Common variants at MS4A4/MS4A6E, CD2AP, CD33 and EPHA1 are associated with late-onset Alzheimer's disease. Nat Genet 43, 436-441, doi:10.1038/ng.801 (2011).
Naj, A. C. et al. Effects of multiple genetic loci on age at onset in late-onset Alzheimer disease: a genome-wide association study. JAMA Neurol 71, 1394-1404, doi:10.1001/jamaneurol.2014.1491 (2014).
Nemes, Z., Devreese, B., Steinert, P. M., Van Beeumen, J., and Fesus, L. (2004). Cross-linking of ubiquitin, HSP27, parkin, and alpha-synuclein by gamma-glutamyl-epsilon-lysine bonds in Alzheimer's neurofibrillary tangles. FASEB J 18, 1135-1137.
Nezis, I. P. et al. Ref(2)P, the Drosophila melanogaster homologue of mammalian p62, is required for the formation of protein aggregates in adult brain. J Cell Biol 180, 1065-1071, doi:10.1083/jcb.200711108 (2008).
Nixon, R. A. New perspectives on lysosomes in ageing and neurodegenerative disease. Ageing Res Rev 32, 1, doi:10.1016/j.arr.2016.11.001 (2016).
Nixon, R. A. The role of autophagy in neurodegenerative disease. Nat Med 19, 983-997, doi:10.1038/nm.3232 (2013).
Nurmohamed, M. T. Newer biological agents in the treatment of rheumatoid arthritis: do the benefits outweigh the risks? Drugs 69, 2035-2043, doi:10.2165/11318290-000000000-00000 (2009).
Pearson, K. Determination of the Coefficient of Correlation. Science 30, 23-25, doi:10.1126/science.30.757.23 (1909).
Perez, S. E. et al. Hippocampal endosomal, lysosomal, and autophagic dysregulation in mild cognitive impairment: correlation with abeta and tau pathology. J Neuropathol Exp Neurol 74, 345-358, doi:10.1097/NEN.0000000000000179 (2015).
Poulose, S. M., Bielinski, D. F. & Shukitt-Hale, B. Walnut diet reduces accumulation of polyubiquitinated proteins and inflammation in the brain of aged rats. J Nutr Biochem 24, 912-919, doi:10.1016/j.jnutbio.2012.06.009 (2013).
Qi, Y. et al. Combined small-molecule inhibition accelerates the derivation of functional cortical neurons from human pluripotent stem cells. Nat Biotechnol 35, 154-163, doi:10.1038/nbt.3777 (2017).
Ray, R. S., Mujtaba, S. F., Dwivedi, A., Yadav, N., Verma, A., Kushwaha, H. N., Amar, S. K.,
God, S., and Chopra, D. (2013). Singlet oxygen mediated DNA damage induced phototoxicity by ketoprofen resulting in mitochondrial depolarization and lysosomal destabilization. Toxicology 314, 229-237.
Reichel, W., Hollander, J., Clark, J. H., and Strehler, B. L. (1968). Lipofuscin pigment accumulation as a function of age and distribution in rodent brain. J Gerontol 23, 71-78.
Reyes, R. C., Brennan, A. M., Shen, Y., Baldwin, Y. & Swanson, R. A. Activation of neuronal NMDA receptors induces superoxide-mediated oxidative stress in neighboring neurons and astrocytes. J Neurosci 32, 12973-12978, doi:10.1523/JNEUROSCI.1597-12.2012 (2012).
Riaz, S., Zeidan, A. & Mraiche, F. Myocardial proteases and cardiac remodeling. J Cell Physiol, doi:10.1002/jcp.25884 (2017).
Rodier, F. & Campisi, J. Four faces of cellular senescence. J Cell Biol 192, 547-556, doi:10.1083/jcb.201009094 (2011).
Rubinsztein, D. C., Marino, G., and Kroemer, G. (2011). Autophagy and aging. Cell 146, 682-695.
Rybicka, J. M., Balce, D. R., Khan, M. F., Krohn, R. M., and Yates, R. M. (2010). NADPH oxidase activity controls phagosomal proteolysis in macrophages through modulation of the lumenal redox environment of phagosomes. Proc Natl Acad Sci USA 107, 10496-10501.
Saitoh, Y. et al. p62 plays a protective role in the autophagic degradation of polyglutamine protein oligomers in polyglutamine disease model flies. J Biol Chem 290, 1442-1453, doi:10.1074/jbc.M114.590281 (2015).
Salminen, A., Kaarniranta, K., Haapasalo, A., Hiltunen, M., Soininen, H., and Alafuzoff, I. (2012). Emerging role of p62/sequestosome-1 in the pathogenesis of Alzheimer's disease. Prog Neurobiol 96, 87-95.
Schneider, J. L. & Cuervo, A. M. Autophagy and human disease: emerging themes. Curr Opin Genet Dev 26, 16-23, doi:10.1016/j.gde.2014.04.003 (2014).
Seidel, K. et al. The p62 antibody reveals various cytoplasmic protein aggregates in spinocerebellar ataxia type 6. Clin Neuropathol 28, 344-349 (2009).
Seredenina, T., Demaurex, N. & Krause, K. H. Voltage-Gated Proton Channels as Novel Drug Targets: From NADPH Oxidase Regulation to Sperm Biology. Antioxid Redox Signal 23, 490-513, doi:10.1089/ars.2013.5806 (2015).
Serrander, L. et al. NOX4 activity is determined by mRNA levels and reveals a unique pattern of ROS generation. Biochem J 406, 105-114 (2007).
Shin, J. (1998). P62 and the sequestosome, a novel mechanism for protein metabolism. Arch Pharm Res 21, 629-633.
Soontornniyomkij, V., Risbrough, V. B., Young, J. W., Soontornniyomkij, B., Jeste, D. V., and Achim, C. L. (2011). Increased hippocampal accumulation of autophagosomes predicts short-term recognition memory impairment in aged mice. Age (Dordr) 34, 305-316.
Spinas, G. A., Bloesch, D., Keller, U., Zimmerli, W. & Cammisuli, S. Pretreatment with ibuprofen augments circulating tumor necrosis factor-alpha, interleukin-6, and elastase during acute endotoxinemia. J Infect Dis 163, 89-95 (1991).
Stout, M. B., Justice, J. N., Nicklas, B. J. & Kirkland, J. L. Physiological Aging: Links Among
Adipose Tissue Dysfunction, Diabetes, and Frailty. Physiology (Bethesda) 32, 9-19, doi:10.1152/physiol.00012.2016 (2017).
Sun, N., Yun, J., Liu, J., Malide, D., Liu, C., Rovira, II, Holmstrom, K. M., Fergusson, M. M., Yoo, Y. H., Combs, C. A., et al. (2015). Measuring In Vivo Mitophagy. Mol Cell 60, 685-696.
Tang, Q. et al. (2012). Gerontologist 52, 290-290.
Tarasoff-Conway, J. M., Carare, R. O., Osorio, R. S., Glodzik, L., Butler, T., Fieremans, E., Axel, L., Rusinek, H., Nicholson, C., Zlokovic, B. V., et al. (2015). Clearance systems in the brain-implications for Alzheimer disease. Nat Rev Neurol 11, 457-470.
Taylor C A, G. S., McGuire L C, Lu H, Croft J B. 2017; Deaths from Alzheimer's Disease—United States, 1999-2014. . MMWR Morb Mortal Wkly Rep 66:521-526. (2017).
Tidball, A. M. et al. Genomic Instability Associated with p53 Knockdown in the Generation of Huntington's Disease Human Induced Pluripotent Stem Cells. PLoS One 11, e0150372, doi:10.1371/journal.pone.0150372 (2016).
Tracy, R. P. Emerging relationships of inflammation, cardiovascular disease and chronic diseases of aging. Int J Obes Relat Metab Disord 27 Suppl 3, S29-34 (2003).
Triplett, J. C., Tramutola, A., Swomley, A., Kirk, J., Grimes, K., Lewis, K., On, M., Rodriguez, K., Cai, J., Klein, J. B., et al. (2015). Age-related changes in the proteostasis network in the brain of the naked mole-rat: Implications promoting healthy longevity. Biochim Biophys Acta 1852, 2213-2224.
Troncoso, J. C. et al. Neuropathology of preclinical and clinical late-onset Alzheimer's disease. Ann Neurol 43, 673-676, doi:10.100²/ana.410430519 (1998).
U. S. Census Bureau DIS. 2012 National Population Projections: Summary Tables. Available at: Accessed May 6. (2017.).
Walston, J. et al. Research agenda for frailty in older adults: toward a better understanding of physiology and etiology: summary from the American Geriatrics Society/National Institute on Aging Research Conference on Frailty in Older Adults. J Am Geriatr Soc 54, 991-1001 (2006).
Wang, M., Song, H., and Jia, J. (2010). Interleukin-6 receptor gene polymorphisms were associated with sporadic Alzheimer's disease in Chinese Han. Brain Res 1327, 1-5.
Wendeln, A. C. et al. Innate immune memory in the brain shapes neurological disease hallmarks. Nature, doi:10.1038/s41586-018-0023-4 (2018).
Westhoff, D. et al. Preoperative cerebrospinal fluid cytokine levels and the risk of postoperative delirium in elderly hip fracture patients. J Neuroinflammation 10, 122, doi:10.1186/1742-2094-10-122 (2013).
Wilcock, D. M., Munireddy, S. K., Rosenthal, A., Ugen, K. E., Gordon, M. N., and Morgan, D. (2004). Microglial activation facilitates Abeta plaque removal following intracranial anti-Abeta antibody administration. Neurobiol Dis 15, 11-20.
Wu, X. et al. IL-4 administration exerts preventive effects via suppression of underlying inflammation and TNF-alpha-induced apoptosis in steroid-induced osteonecrosis. Osteoporosis international: a journal established as result of cooperation between the European Foundation for Osteoporosis and the National Osteoporosis Foundation of the USA 27, 1827-1837, doi:10.1007/s00198-015-3474-6 (2016).
Wyss-Coray, T., Loike, J. D., Brionne, T. C., Lu, E., Anankov, R., Yan, F., Silverstein, S. C., and Husemann, J. (2003). Adult mouse astrocytes degrade amyloid-β in vitro and in situ. Nature Medicine 9, 453-457.
Xia, X. et al. Glucocorticoid-induced autophagy in osteocytes. Journal of bone and mineral research: the official journal of the American Society for Bone and Mineral Research 25, 2479-2488, doi:10.1002/jbmr.160 (2010).
Xu, M. et al. JAK inhibition alleviates the cellular senescence-associated secretory phenotype and frailty in old age. Proc Natl Acad Sci USA 112, E6301-6310, doi:10.1073/pnas.1515386112 (2015).
Yao, W. et al. Sclerostin-antibody treatment of glucocorticoid-induced osteoporosis maintained bone mass and strength. Osteoporosis international: a journal established as result of cooperation between the European Foundation for Osteoporosis and the National Osteoporosis Foundation of the USA 27, 283-294, doi:10.1007/s00198-015-3308-6 (2016).
Yao, W., Dai, W., Jiang, J. X. & Lane, N. E. Glucocorticoids and osteocyte autophagy. Bone 54, 279-284, doi:10.1016/j.bone.2013.01.034 (2013).
Ye, S. M. & Johnson, R. W. Increased interleukin-6 expression by microglia from brain of aged mice. J Neuroimmunol 93, 139-148 (1999).
Yerbury, J. J., Ooi, L., Dillin, A., Saunders, D. N., Hatters, D. M., Beart, P. M., Cashman, N. R., Wilson, M. R., and Ecroyd, H. (2016). Walking the tightrope: proteostasis and neurodegenerative disease. J Neurochem 137, 489-505.
Yin, K. J., Cirrito, J. R., Yan, P., Hu, X., Xiao, Q., Pan, X., Bateman, R., Song, H., Hsu, F. F., Turk, J., et al. (2006). Matrix metalloproteinases expressed by astrocytes mediate extracellular amyloid-beta peptide catabolism. J Neurosci 26, 10939-10948.
Yip, Y. P., Capriotti, C., Magdaleno, S., Benhayon, D., Curran, T., Nakajima, K., and Yip, J. W. (2004). Components of the reelin signaling pathway are expressed in the spinal cord. J Comp Neurol 470, 210-219.
Zhang, J. et al. Microglial CR3 activation triggers long-term synaptic depression in the hippocampus via NADPH oxidase. Neuron 82, 195-207, doi:10.1016/j.neuron.2014.01.043 (2014).
Zhang, X., Cheng, X., Yu, L., Yang, J., Calvo, R., Patnaik, S., Hu, X., Gao, Q., Yang, M., Lawas, M., et al. (2016). MCOLN1 is a ROS sensor in lysosomes that regulates autophagy. Nature communications 7, 12109.
Zhu, Y. et al. Inflammation and the depot-specific secretome of human preadipocytes. Obesity (Silver Spring) 23, 989-999, doi:10.1002/oby.21053 (2015).

1. A method of treating a neurodegenerative disease, fibrotic disease, or rheumatological disease in a subject comprising administering to the subject a therapeutic agent; wherein the therapeutic agent inhibits nicotinamide adenine dinucleotide phosphate (NADPH)-oxidase (NOX) or a downstream NOX effector.

2. The method of claim 1, wherein the neurodegenerative disease comprises Alzheimer's disease, Parkinson's disease, Huntington's disease, spinocervellar ataxia type 1, age-related dementia, lewy body dementia, probably vascular dementia, frontotemporal dementia, and amyotrophic lateral sclerosis (ALS).

3. The method of claim 1, wherein the NOX is a NOX isoform selected from NOX1, NOX2, NOX3, NOX4, or NOX5

4. The method of claim 1, wherein the therapeutic agent is a signal transducer and activator of transcription 3 (STAT3) inhibitor, and wherein the therapeutic agent reduces STAT3 mediated NOX activation, thereby causing NOX inhibition.

5. The method of claim 1, wherein the therapeutic agent is an anti-IL-6 antibody or anti-inflammatory agent, and wherein the therapeutic agent reduces IL-6 mediated NOX activation, thereby causing NOX inhibition.

6. The method of claim 1, wherein the downstream NOX effector comprises reactive oxygen species, hydrogen peroxide, or superoxide.

7. The method of claim 1, wherein the therapeutic agent that is a small molecule, antibody, siRNA, antisense oligonucleotide, peptide, or protein.

8. The method of claim 7, wherein the therapeutic agent is a small molecule comprises diphenylene iodonium, Apocynin, 2-(2-chlorophenyl)-4-[3-(dimethylamino)phenyl]-5-methyl-1H-pyrazolo [4,3-c]pyridine-3,6-dione (GKT-831), Rapamycin, dismutazyme, a malonic acid derivative of C60, an acetic acid derivative of C60, or any combination thereof.

9. The method of claim 1, wherein the agent is administered prior to the onset of pathological changes characteristic of a neurodegenerative disease.

10. A method of reducing age-dependent lysosome impairment and/or increasing initiation of autophagy in a subject comprising administering to the subject a therapeutic agent; wherein the therapeutic agent inhibits nicotinamide adenine dinucleotide phosphate (NADPH)-oxidase (NOX) or downstream NOX effector.

11. The method of claim 10, wherein the lysosome impairment is present in hippocampal pyramidal neurons and parvalbumin interneurons.

12. The method of claim 10, wherein the NOX is a NOX isoform selected from NOX1, NOX2, NOX3, NOX4, or NOX5

13. The method of claim 10, wherein the downstream NOX effector comprises reactive oxygen species, hydrogen peroxide, or superoxide.

14. The method of claim 10, wherein the therapeutic agent is a signal transducer and activator of transcription 3 (STAT3) inhibitor, and wherein the therapeutic agent STAT3 mediated NOX activation, thereby causing NOX inhibition.

15. The method of claim 10, wherein the therapeutic agent is an anti-IL-6 antibody or anti-inflammatory agent, and wherein the therapeutic agent reduces IL-6 mediated NOX activation, thereby causing NOX inhibition.

16. The method of claim 10, wherein the therapeutic agent is a small molecule, antibody, siRNA, antisense oligonucleotide, peptide, or protein.

17. The method of claim 16, wherein the agent is a small molecule comprises diphenylene iodonium, Apocynin, 2-(2-chlorophenyl)-4-[3-(dimethylamino)phenyl]-5-methyl-1H-pyrazolo[4,3-c]pyridine-3,6-dione (GKT-831), Rapamycin, dismutazyme, a malonic acid derivative of C60, an acetic acid derivative of C60, or any combination thereof.

18. A method of improving lysosome function in a subject comprising administering to the subject a therapeutic agent; wherein the therapeutic agent reduces reactive oxygen species (ROS), hydrogen peroxide, or superoxide.

19. The method of claim 18, wherein the agent that improves lysosome function by inhibiting ROS, hydrogen peroxide, or superoxide is a small molecule, antibody, siRNA, antisense oligonucleotide, peptide, or protein.

20. The method of claim 19, wherein the agent is a small molecule comprises dismutazyme, a malonic acid derivative of C60, an acetic acid derivative of C60, or any combination thereof.
